(19) United States
(12) Patent Application Publication
(10) Pub. No.: US 2010/0047341 A1
(43) Pub. Date: Feb. 25, 2010

(54) COMBINED PREPARATION FOR THE TREATMENT OF CARDIOVASCULAR DISEASES BASED ON CHRONOTHERAPY THEORY

(75) Inventors: Sung Wuk Kim, Seongnam-si (KR); Sung Soo Jun, Seongnam-si (KR); Young Gwan Jo, Daejeon (KR); Ja-Seong Koo, Daejeon (KR); Sang Ouk Sun, Gwangju (KR)

Correspondence Address: ROTHWELL, FIGG, ERNST & MANBECK, P.C., 1425 K STREET, N.W., SUITE 800, WASHINGTON, DC, 20005, US

(73) Assignee: HANALL PHARMACEUTICAL CO., LTD., Daejeon (KR)

(21) Appl. No.: 12/445,204

(22) PCT Filed: Oct. 10, 2007
(86) PCT No.: PCT/KR07/04929

§ 371 Date: Sep. 17, 2009

(30) Foreign Application Priority Data
Oct. 10, 2006 (KR) 10-2006-0098438
Oct. 9, 2007 (KR) 10-2007-0101492

(51) Int. Cl. A61K 9/54; A61K 9/24; A61K 31/4178

(52) U.S. Cl. 424/458; 424/472; 514/381

(57) ABSTRACT

The present invention relates to a functional combination preparation comprising a dihydropyridine-based calcium channel blocker such as amlodipine and an ARB (Angiotensin-2 receptor blocker) such as losartan. In particular, the present invention relates to a chronotherapeutical combination pharmaceutical formulations with controlled-release for the prevention or treatment of cardiovascular disease, which is formulated in accordance with xenobiotics and chronotherapy for enabling the two drugs to be chronotherapeutically released, thereby improving the therapeutic activity as compared to the co-administration of each drug in the form of a single pill, while reducing side effects and maintaining the therapeutic activity as high as possible at the time of day when the risk of a complication of cardiovascular disease is highest.

### TECHNICAL FIELD

The present invention relates to pharmaceutical combination formulations comprising a dihydropyridine-based calcium channel blocker such as amlodipine and an ARB (angiotensin-2 receptor blocker) such as losartan. In particular, the present invention relates to chronotherapeutically designed combination formulations for the prevention and treatment of cardiovascular diseases, which is formulated based on xenobiotics and chronotherapy for enabling the two drugs to be chronotherapeutically released, thereby improving the therapeutic activity as compared to the co-administration of each drug in the form of a single tablet, while reducing side effects and maintaining the therapeutic activity as high as possible during the period of time of a day when the risk of a complication of cardiovascular disease is highest.

### BACKGROUND ART

Although numerous anti-hypertensive agents with excellent efficacies have been developed and prescribed recently, there still remains 50% Rule with regard to the treatment of hypertension. That is, only 50% of hypertension patients recognize that they have the symptom, and only 50% of those who acknowledge their disorder receive medical care, where only 50% of them are subject to appropriate medical treatment. This means that only 12.5% of the hypertension patients receive proper medical treatment.

In particular, anti-hypertensive therapy aims not only to lower blood pressure but also to prevent complications such as myocardial infarction, cardiac insufficiency, stroke and early death or to control their medical inadequacy, thereby securing long and healthy lives of those patients.

To achieve the aforementioned goals, the anti-hypertensive agents should be further improved to facilitate the medication instruction and increase patient's compliance.

Large-scale clinical reports for the past 30 years (e.g., HOT, UKPOS) have proved that a combination prescription may prevent the complications or suppress their aggravation when prescribed to patients suffering from a minor or moderate hypertension [Guidelines for hypertension management issued by Joint National Committee (JNC VI & VII), WHO-ISH (1999)].

There are various causes for hypertension. Each cause may be responsible for initial hypertension of a patient, but multiple causes may be involved in the hypertension of the patient in the end. Thus, it is difficult to choose a certain antihypertensive drug to be suitable for the incumbent pathophysiological cause of a hypertensive patient. [Journal of human hypertension 1995: 9: S33-S36]. For this reason, a combination therapy of anti-hypertensive agents has been preferred and on the increase, in particular, based on ARB (Angiotensin-2 Receptor Blockers) drugs such as losartan.

A combination therapy has frequently been reported as necessary for the following reasons [J. Hum. Hypertens. 1995: S33-S36].

1) Hypertension is eventually aggravated by multiple causes and factors, although it may be triggered by a single cause.

2) A single active ingredient is not suitable for multiple pathophysiological causes of hypertension.

3) A single active ingredient is only effective to less than 50% of patients.

4) A combination prescription is effective to more than 80% of patients.

5) In particular, by prescribing only a single pill, it is difficult to treat hypertension with complications such as diabetes, and even more difficult to prevent the aggravation of complications.

6) When the dose is increased because a single pill is not satisfactory in efficacy, side effect may be increased. A combination prescription may reduce the side effects.

7) A combination prescription may remove the various causes of diseases while preventing complications and reducing side effects. Therefore, American Heart Association also emphasizes that a combination prescription may be preferably recommended to a single pill prescription to start the hypertension treatment

8) In particular, blood pressure should be much lowered in patients with complications than those without. A combined therapy is essential in this case. Nevertheless, a single pill may be effective only for 26% of patients. A combination preparation may be effective for as many as 74% of patients in preventing complications by maintaining proper blood pressure [Large-scaled clinical test, HOT].

9) FDA has been acknowledging for 30 years the necessity of a combined preparation in accordance with a fixed-dose combination therapy. According to this principle, it is required that drugs with different pharmaceutical activities be combined to contain the same amounts, as if it were taken in each single pill. This is called a fixed-dose combination preparation, and has been approved without further experimental data if the efficacy and safety of the single pills are fully evaluated and such combination therapy has been widely prescribed by doctors.

10) It is well known that a fixed-dose combination of anti-hypertensive agents has excellent effect in lowering blood pressure.

11) Dose of each ingredient is not increased, and thus the side effects of each ingredient are greatly reduced.

12) Anti-hypertensive agents cause side effects mainly related to circulatory system. Such side effects may be more aggravated by increasing the dose of a single agent rather than by combination therapy of two drugs without increasing the dose of each drug.

13) A combination preparation may improve the medication compliance, and save a half of the time required for doctors to educate the patients with the medication instruction.

14) A combination therapy preparation may lower the risk of circulatory complications, and reduce the long-term expenses to be incurred for the disease control.

15) A combination product may reduce significantly the expenses and time required for packing comapared with those for two of each single product.

In general, blood pressure increases with age. About 63% of elderly people above age of 60 suffer from hypertension. In particular, isolated systolic hypertension occurs around the age of 60 as systolic blood pressure increases while diastolic blood pressure decreases, which is called as geriatric hypertension.

In the mean time, renal function of hypertensive patients declines with age. For this reason, the production of blood pressure raising factor (angiotensin II) increases, thereby further elevating the blood pressure. In this case, a single pill may not bring in satisfactory results, and a combination therapy of the amlodipine and the losartan effective in protection of the kidney has been reported as an effective medication [Clin. Ther. 2003 May 25 (5): 1469-69; Nepherol Dial Transplant vol. 18(2003): p1806-1813; J. American Society of Nephrology, vol. 12 (2001) p. 822-827].

A constant level of blood pressure is required to be maintained for 24 hours in geriatric hypertension. Further, it is also necessary to prevent sudden heart attack that may occur during sleep and hypertensive stroke caused by stress during the early part of the daytime.

Each anti-hypertensive agent shows a unique circardian biorhythmic activity. Losartan, an ARB drug, shows a remarkable anti-hypertensive effect which occurs from midnight to dawn when RAAS is activated strongly. Amlodipine, a dihydropyridine-based calcium channel blocker, favorably suppresses the hypertension caused by stress vasospasm when a patient is awake.

Therefore, therapeutic activity may be further increased by combination of these drugs [Clin. Hypertension 5(1): 17-23, 30, 2003].

A combination therapy may be ideal because the pharmaceutical activity of each drug is different as presented in Table 1 below.

TABLE 1
Pharmacological activity of amlodipine and losartan

| | Amlodipine | Losartan |
|---|---|---|
| Vasodilation | Relaxes blood vessel by | Relaxes blood vessel by |
| | decreasing the inflow of | suppressing aldosterone activity |
| | calcium ions into vascular | and by blocking angiotensin-2 |
| | smooth muscle cells | activity |
| Circardian | Favorably effective in | More strongly suppressing |
| Biorhythmic action | vasospasm due to stress | RAAS more activated after |
| | during awakend daytime | midnight and more favorably |
| | | effective in non-dipper |
| | | hypertension* |
| Influence on | Causes the loss of K⁺ | Suppresses the loss of K⁺ |
| electrolyte | | |
| Action on renin | Very effective in hypo-renein | Very effective in hyper-renein |
| | patients | patients |
| Influence on | Favorably acting on | Helpful in increasing insulin |
| diabetics | insulinemia of the type 2 | sensitivity of diabetes |
| | diabetes and improving | |
| | glucose metabolism | |
| Impact on | Suppressing the atheromic | Inhibiting the proliferation of |
| atherosclerosis and | proliferation in blood vessel | pathogenic cells by suppressing |
| hyperlipidemia | wall | the stimulus of angiotensin-2 on |
| (and complication) | Suppressing the | blood vessel wall |
| | overproduction of connective | |
| | tissue in blood vessel wall | |
| | Suppressing the peroxidation | |
| | of LDL | |
| | Lowering plasma | |
| | triglycerides | |
| Vascular | Stimulating the regeneration | Stimulating the regeneration |
| endothelial | and maintenance of vascular | and maintenance of vascular |
| function | endothelial cells | endothelial cells |
| Vasodilating action | Acitng on afferent vessel | Acitng on afferent vessel |
| on glomerular | | |
| artery | | |
| Albuminuria | Lowered | Lowered |
| Proliferation of | Suppressed | Suppressed |
| kidney mesangial | | |
| cell | | |

 RASS (Renin and Angiotensin System): One of mechanism for regulating blood pressure in a body
 Non-dipper hypertension: Hypertension where blood pressure does not decrease in one's sleep unlike normal people. Frequently found in aged people and patients suffering from diabetes or cardiac hypertrophy. Relatively high risk of complications such as stroke.

That is, when the two drugs are administered at certain time intervals, a combination therapy of the two drugs may show a synergistic effect in controlling hypertension for a patient, to whom the efficacy of single drug is insufficient, because the two drugs are different from each other in the mechanism in lowering blood pressure and the time of maximum activity. Further, the two drugs are complementary action in that losartan compensates the loss of potassium ion induced by amlodipine. Furthermore, chronotherapeutical combination pharmaceutical formulations with controlled-release may reduce the insulin resistance, which often occurs to patients suffering from type 2 diabetes.

Amlodipine, i.e., 3-ethyl-5-methyl2-(2-aminoethoxymethyl)-4-(2-chlorophenyl)-1,4-dihydro-6-methyl-3,5-pyridinedicarboxylate, is a long acting calcium channel blocker. Its effectivity and safety may be proven by the fact that amlodipine is the most widely prescribed drug for the treatment of hypertension [European patent application publication No. 89,167 and U.S. Pat. Nos. 4,572,909, 4,879,303 and 5,115,120].

Losartan, i.e., 2-butyl-4chloro-1-[p-(o-1H-tetrazol-5-ylphenyl)benzyl]imidazole-5-methanol, suppresses the increase in blood pressure by inhibiting angiotensin II (AII) from binding with AII receptor (blood vessel wall receptor). The angiotensin-II induces the increase in blood pressure, hypertrophy of left ventricle or blood vessel, atherosclerosis, renal insufficiency and stroke [U.S. Pat. No. 5,138,069].

Due to the activity of lowering blood pressure, losartan is widely used for prevention or treatment of various diseases such as cardiac insufficiency, arrhythmia or cardiac insufficiency with atherosclerosis, diabetes-related complications, stroke, atherosclerosis and cardiovascular circulatory disorders, or for anti-platelate activity, or for the inhibition of harmful activity of aldosterone or influence of metabolic syndrome [Clin. Exp. Hypertens., vol. 20 (1998), p. 205-221; J. Hypertens., vol. 13 (8) (1995), p. 891-899; Kidney Lnt., vol. 57(2)(2000), p. 601-606; Am. J. Hypertens., vol. 10 (12PT2) Suppl. (1997), p. 325-331; Circulation, vol. 101 (14) (2000), p. 1653-1659; J. Hypertension., vol. 17 (7) (1999), p. 907-716; Circulation, vol. 101 (2000), p. 2349].

Amlodipine and losartan act widely and complementarily as shown in Table 1, and thus they are considered as a target of a combination prescription or therapy. The combination prescription or therapy of amlodipine and losartan is also effective for various diseases. For these reasons, a combination therapy (co-administration of each single pill of the two drugs), has been widely attempted.

However, the two drugs act with different circardian biorhythmic pattern, and it is known by the xenobiotics that the two drugs may be affected by or act on a certain enzyme in an opposite manner when released and absorbed in the liver at the same time [Cytochrome P450 Drug Interaction Table, Department of Medicine, Indiana University updated 2004 Mar. 11].

The so-called xenobiotics is the scientific study for developing a drug efficacy for each individual by examining the metabolism process of drugs in a body to determine appropriate medication time, to prevent the antagonism of drugs by making the differentiation in absorption time of the drugs, and to investigate the enzyme metabolism of each individual.

In this respect, the following problems may be caused when amlodipine and losartan are co-administered two different dosage forms.

Losartan is absorbed and transported to the liver. Some amount of losartan itself as active form is immediately released from the liver to blood, reaching the maximum level as its form within an hour. However, all the remaining losartan compounds are metabolized mostly by two enzymes in the liver (cytochrome P450 2C7 and 3A4), to be converted into the activated form (losartan carboxylate). Maximum plasma concentration can be reached within 3-4 hours after the conversion. That is, a mixture of losartan and losartan carboxylate shows the total pharmaceutical activity of losartan.

Meanwhile, amlodipine inhibits the generation of cytochrome P450 3A4 in the liver. Amlodipine is itself an active form, and at the presence of cytochrome P450 3A4, a part of amlodipine in the liver is metabolized, but most of amlodipine are inhibiting the further generation of cytochrome P450 3A4. Therefore, 60-90% of amlodipine in its active form may show maximum plasma concentration at the time of 6-12 hours.

Therefore, when amlodipine is absorbed in the liver at the same time with losartan or within 3-4 hours before losartan is absorbed, the conversion of losartan into its active metabolite (losartan carboxylate) may be inhibited. In particular, isolated systolic blood pressure in an aged people may cause stroke, even although such isolated systolic blood pressure is slightly elevated. Therefore blood pressure should be maintained at the normal level strictly for 24 hours to prevent from complications. However, no combination preparation that may overcome the aforementioned clinical drawbacks has been reported.

Osvaldo Kohlmann et al. have reported the necessity of combination therapy and experimental result based on the result that compared the single therapy of amlodipine or losartan with a co-administration prescription of amlodipine and losartan. However, this is different from the present invention which is a combination predict technically formulated to play with the functional differences. [Evaluation of efficacy and tolerability of the fixed combination of amlodipine and losartan in the treatment of essential hypertension, J. Cardiol. 2006].

U.S. patent application publication 2005-0209288 describes that the combined administration therapy of (S)-type amlodipine and telmisartan (i.e., ARB drug such as losartan U.S. Pat. No. 5,591,762) may achieve the same efficacy even at a lower dose as compared to the separate administration of each drug. However, this publication fail to consider the fact that a formulation should comprise a basic agent for effective plasma concentration and therapeutic activity due to the physical property of telmisartan [Korean patent publication No. 2005-0053690]. Further, this publication considers only necessity of the co-administration of single pill (i.e., a co-administratin prescription method), and fail to attempt of preparing a combination product to be efficacious therapies of cardiovascular diseases.

U.S. patent application publication No. 2003-0158244 discloses a formulation comprising losartan that stays in the stomach after oral administration and is gradually released, a preparation comprising this formulation, and a combined preparation further comprising an anti-hypertensive agent. This publication describes that the formulation or preparation therein may reduce the side effects caused by overdose by inducing an appropriate absorption of drug, delaying the time at maximum plasma concentration and lowering the maximum plasma concentration. However, this technique may not achieve the therapeutic efficacy herein because the principle of this technique is the opposite to that of the present invention (xenobiotics and chronotherapy).

Korean patent publication No. 2004-0078140 discloses an anti-hypertensive combination preparation comprising valsartan (ARB drug) and calcium channel blocker. However, the effect that two drugs are released at certain time intervals may not be achieved in this invention.

WO 06/048208 discloses a double-layered tablet comprising telmisartan and amlodipine. This publication selected a double-layered tablet as a formulation for improving the stability of an active ingredient, and the formulation is designed so that both the two ingredients may be disintegrated rapidly. This technique is totally different from the present invention where the release of calcium channel blocker such as amlodipine is delayed according to xenobiotics and chronotherapy. Further, it is obvious that there is no inventive step in the above technique because it failed to consider the characteristics of the drug.

### DISCLOSURE

### Technical Problem

To overcome the aforementioned problems, the present inventors have exerted extensive research and finally developed a functional combination product where an ARB drug such as losartan is absorbed from the small intestine immediately after the administration while a dihydropyridine-based calcium channel blocker such as amlodipine is absorbed from the small intestine 3-4 hours after the administration, thereby it is possible to maintain constant blood pressure for 24 hours and inhibit complications and other side effects by the administration once a day in the evening. The functional combination preparation according to the present invention has the advantages as in Table 2.

TABLE 2
Advantages of the functional combination preparation
according to the present invention over the conventional
co-administraion prescription

| | |
|---|---|
| 1) | Excellent in lowering blood pressure (15% higher than the |
| | commercially available preparation, Experimental Example 2 of |
| | Hanall Pharmaceutical Co., Ltd.) |
| 2) | Excellent in inhibiting side effects |
| 3) | Maximized activity in the time of a day when the risk of a |
| | complication is highest. |
| 4) | Appropriate for patients showing non-dipper hypertension, |
| | whose risk of complications is comparatively higher. |
| 5) | Reduction in time required for medication instructions |

Therefore, the present invention aims to provide a combination drug system and a functionally designed combination preparation that may maximize the pharmaceutical and clinical efficacy in the treatment of hypertension and in the prevention of its complications or other side effects as compared to the co-administration of a dihydropyridine-based calcium channel blocker single pill and an ARB single pill.

The present invention also aims to open a new aera of the functional combination product formulated by the DDS technology, which applies the theory of xenobiotics and chronotherapy in the conventional fixed-dose combination preparation.

Further, the present invention aims to maximize the overall therapeutic efficacy of the two or more drugs and facilitate the medication compliance of aged people by simplifying the medication in such a way as once a day in the evening. Furthermore, the present invention aims to reduce the expenses and time required for packing individually single pills or making up a prescription, which increases twice or more than for the co-administration of two or more drugs.

### Technical Solution

The present invention a chronotherapeutical combination pharmaceutical formulations with controlled-release comprising a dihydropyridine-based calcium channel blocker and an ARB (angiotensin-2 receptor blocker) as active ingredients, where the ARB (angiotensin-2 receptor blocker) is immediately released while the dihydropyridine-based calcium channel blocker is gradually released after some lag time.

### Advantageous Effects

A functional combination preparation according to the present invention applies the xenobiotics and the chronotherapy to the drug formulation technique, thereby fully achieving the pharmaceutically or clinically therapeutic efficacy that may be lost by the co-administration of the amlodipine single pill and the losartan single pill. The functional combination preparation herein may also show constant activities in controlling the blood pressure, in preventing the complications, and lessening side effects. Further, the functional combination preparation herein may improve the medication compliance of the aged people compared with that of the simplified co-administration.

Further, it is expected that a combination preparation will exert the increased efficacy in the treatment of the mild hypertension up to about 80%, as compared with about 50% in case of each single pill. It will contribute to the healthy longevity of the hypertensive patients that the functional combination preparation of the present invention shows remarkable efficacy in such major complications such as heart diseases, kidney diseases and stroke. In particular, a functional combination preparation will be the best prescription or therapy for a hypertensive patient suffering from diabetes complication.

Further, the two drugs in a functional combination preparation herein have different activities and reduce the side effects of each drug, and also lower the risk of circulatory complications. The present invention is also efficient in economical respect in that a combination prescription will curtail the long-term expenses to be incurred by complications, which might be aggravated by less effective therapy of the conventional co-administration, and will save the packaging cost and the time for the prescription.

Therefore, the present invention will open the new aera of the functional combination product formulated by the DDS technology, which applies the theory of xenobiotics and chronotherapy in the conventional fixed-dose combination preparation.

### DESCRIPTION OF DRAWINGS

FIG. 1 shows the results of Experimental Example 1, i.e., the dissolution rates of losartan and amlodipine in a losartan single pill(Cozaar® 50 mg tablet—MSD Korea), an amlodipine single pill(Norvasc® 5mg tablet—Pfizer) and the functional combination preparation prepared in Example 4.

FIG. 2 shows the results of Experimental Example 1, i.e., the dissolution rates of amlodipine in the functional combination preparation prepared in Examples 4 and 8-10.

FIG. 3 shows the results of Experimental Example 1, i.e., the dissolution rates of valsartan and amlodipine in a valsartan single pill (Diovan® 80 mg tablet Korea Novartis), an amlodipine single pill (Norvasc® 5 mg tablet—Pfizer) and the functional combination preparation prepared in Example 11.

FIG. 4 shows the results of Experimental Example 1, i.e., the dissolution rates of telmisartan and amlodipine in a telmisartan single pill (Pritor® 40 mg tablet GSK), an amlodipine single pill (Norvasc® 5mg tablet—Pfizer) and the functional combination preparation prepared in Example 12.

FIG. 5 shows the results of Experimental Example 1, i.e., the dissolution rates of lercanidipine and losartan in a lercanidipine single pill (Zanidip® tablet—LG Life Science), a losartan single pill (Cozaar® 50 mg tablet—MSD Korea) and the functional combination preparation prepared in Example 16.

FIG. 6 shows the results of Experimental Example 1, i.e., the dissolution rates of lacidipine and losartan in a lacidipine single pill (Zanidip® tablet—LG Life Science), a losartan single pill (Cozaar® 50 mg tablet—MSD Korea) and the functional combination preparation prepared in Example 27.

FIG. 7 shows the results of Experimental Example 1, i.e., the dissolution rates of amlodipine besylate and losartan in an amlodipine besylate single pill (Norvasc® 5 mg tablet—Pfizer), losartan single pill (Cozaar® 50 mg tablet—MSD Korea) and the functional combination preparation prepared in Example 18.

FIG. 8 shows the results of Experimental Example 1, i.e., the dissolution rates of amlodipine in the functional combination preparation prepared in Examples 2-4.

FIG. 9 shows the systolic blood pressure (SBP) within 20 hours after the administration of drugs according to Experimental Example 2.

FIG. 10 shows the mean blood pressure (MBP) within 20 hours after the administration of drugs according to Experimental Example 2.

FIG. 11 shows the diastolic blood pressure (DBP) within 20 hours after the administration of drugs according to Experimental Example 2.

### BEST MODE

The present invention relates to a combination preparation preparation comprising a dihydropyridine-based calcium channel blocker and an angiotensin-2 receptor blocker (ARB) as active ingredients, wherein the angiotensin-2 receptor blocker (ARB) is rapidly released and then the dihydropyridine-based calcium channel blocker is gradually released with some lag time.

The present invention also relates a combination preparation for the treatment of cardiovascular disease, which comprises:

(1) an immediate-release granule comprising the angiotensin-2 receptor blocker (ARB) as an active ingredient; and

(2) a delayed-intermediate-release granule or coated tablet comprising the dihydropyridine-based calcium channel blocker as an active ingredient and a release-controlling material selected from the group consisting of a water-soluble polymer, a water-insoluble polymer, an enteric polymer and a mixture thereof.

Hereunder is provided a detailed description of the present invention. As compared to the co-administration of a single pill of a dihydropyridine-based calcium channel blocker (e.g., amlodipine) and a single pill of an ARB (e.g., losartan), a functional combination preparation for the treatment of cardiovascular disease according to the present invention maximizes the overall therapeutic efficacy of two or more drugs, which maintains constant activities for 24 hours in the treatment of hypertension and in the prevention of its complication by allowing the two drugs to be released at a predetermined time interval.

The present invention relates to a combination drug system or a functional combination preparation, where the ARB (angiotensin-2 receptor blocker) is released immediately after the administration while the dihydropyridine-based calcium channel blocker is released after some lag time. Preferably, a combination preparation herein comprises (i) an immediate-release part containing ARB (angiotensin-2 receptor blocker) as active ingredients; and (ii) a delayed-intermediate-release part containing dihydropyridine-based calcium channel blocker as active ingredients and a release-controlling material selected from the group consisting of water-soluble polymer, water-insoluble polymer, enteric polymer and a mixture thereof.

Examples of the dihydropyridine-based calcium channel blocker include amlodipine, lercanidipine, felodipine, nifedipine, nicardipine, isradipine, nisoldipine and a pharmaceutically acceptable salt thereof. Examples of the ARB (angiotensin-2 receptor blocker) include losartan, valsartan, telmisartan, irbesartan, candesartan, olmesartan and a pharmaceutically acceptable salt thereof.

The present invention discloses a formulation technique comprising (i) granules or a coated tablet containing dihydropyridine-based calcium channel blocker prepared by effectively press-formulating into a matrix for intentionally delaying the release of an active ingredient but immediately releasing the ingredient at the release time; and (ii) immediate-release granules containing ARB(s) for intended to make presscoated tablets, triple layered tablets and multicomponent matrix type tablets respectively.

That is, the present invention relates to a functional combination preparation for the treatment of cardiovascular disease, which comprises (1) an immediate-release granule comprising an ARB (angiotensin-2 receptor blocker) as active ingredients; (2) a delayed-immediate-release granule or coated tablet comprising a dihydropyridine-based calcium channel blocker as active ingredients and a release-controlling material selected from the group consisting of water-soluble polymer, water-insoluble polymer, enteric polymer and a mixture thereof.

The aforementioned granules containing the dihydropyridine-based calcium channel blocker comprises a release-controlling material selected from the group consisting of water-soluble polymer, water-insoluble polymer and enteric polymer, and may be coated according to the conventional method. The resulting granules or coated tablets may be compressed into a tablet along with multi-component particles or granules of an immediate-release granule composition containing ARB, or may be filled in a capsule.

A combination preparation herein may be formulated into multi-layered tablet comprising (i) a dihydropyridine-based calcium channel blocker granule that is immediately released after some lag time; and (ii) an ARB granule layer that is immediately released.

A combination preparation herein may also be formulated into a double inner core tablet comprising (i) an inner core layer containing a dihydropyridine-based calcium channel blocker that is immediately released after some lag time; and (ii) an outer layer containing ARB that is immediately released.

The present invention discloses a technique for preparing an immediate-release ARB layer so as to be rapidly disintegrated, released and absorbed in the gastric tract after oral administration, separately from the layer containing dihydropyridine-based calcium channel blocker, by using a pharmaceutically acceptable additive such as a filler, a binder, a disintegrant, a lubricant, a stabilizer and a film coating agent.

The present invention discloses a formulation technique enabling the delayed-immediate-release of an active ingredient. By means of this technique, less than 20% of the active ingredient is released until an intended time, typically within 1-6 hours after oral administration, while 85% or more of the ingredient is released after the intended time. The present invention discloses a delayed-immediate-release preparation of dihydropyridine-based drugs such as amlodipine. Accordingly, an ARB drug layer containing losartan is first released and absorbed, and a dihydropyridine-based calcium channel blocker containing amlodipine is released and absorbed 1-6 hours, preferably 3-4 hours, later.

This delayed-immediate-release preparation may be prepared by using an active ingredient, a polymeric material that allows the delayed-immediate-release behavior and other additives such as a pharmaceutically acceptable filler, a binder, a disintegrant, a lubricant and a stabilizer.

In the present invention, the ARB (angiotensin-2 receptor blocker) is preferred to be contained in the amount of 0.2-20 weight parts, more preferably 2-12 weight parts, relative to one weight part of the dihydropyridine-based calcium channel blocker. When the amount is less than 0.2 weight parts, the desired hypertensive effect may not be sufficient. When the amount is more than 20 weight parts, there may be side effects such as low blood pressure.

In the present invention, a delayed-intermediate-release part may comprise the release-controlling material in the amount of 0.5-100 weight parts, preferably 1-50 weight parts, most preferably 2-30 weight parts relative to one weight part of the dihydropyridine-based calcium channel blocker. When the amount is less than 0.5 weight parts, retention time may not be sufficient. When the amount is more than 100 weight parts, drugs may not be released or retention time may exceed 12 hours.

Examples of the water-soluble polymer include but are not limited to water-soluble cellulose ester selected from the group consisting of methylcellulose, hydroxypropyl cellulose and hydroxypropylmethylcellulose; water-soluble polyvinyl derivative selected from the group consisting of polyvinylpyrrolidone and polyvinylalcohol; alkylene oxide polymer selected from the group consisting of polyethyleneglycol and polypropyleneglycol; and a mixture thereof.

Examples of the water-insoluble polymer include but are not limited to water-insoluble cellulose ether selected from the group consisting of ethylcellulose and cellulose acetate; water-insoluble acrylic acid based copolymer selected from the group consisting of acrylic acid ethyl.methacrylic acid methyl.methacrylic acid chlorotrimethylammonium ethyl copolymer (e.g., Eudragit™ RS or RL, Degussa) and methacrylic acid methyl.acrylic acid ethyl copolymer chlorotrimethyl ammonium ethyl copolymer (e.g., Eudragit™ NE30D, Degussa); and a mixture thereof.

Examples of the enteric polymer include but are not limited to enteric cellulose derivatives selected from the group consisting of hydroxypropylmethylcellulose acetate succinate, hydroxypropylmethylcellulose phthalate, hydroxymethylethylcellulose phthalate, cellulose acetate phthalate, cellulose acetate succinate, cellulose acetatemaleate, cellulose benzoate phthalate, cellulose propionate phthalate, methylcellulose phthalate, carboxymethylethylcellulose and ethylhydroxyethylcellulose phthalate; enteric acrylic acid based copolymer selected from the group consisting of styrene.acrylic acid copolymer, acrylic acid methyl.acrylic acid copolymer, acrylic acid methylmethacrylic acid copolymer, acrylic acid butyl.styrene.acrylic acid copolymer, methacrylic acid.methacrylic acid ethyl copolymer (e.g., Eudragit™ L 100, Eudragit™ S, Degussa), methacrylic acid.acrylic acid ethylcopolymer (e.g., Eudragit™ L 100-55, Degussa) and acrylic acid methyl.methacrylic acid.acrylic acid octyl copolymer; enteric maleic acid based copolymer selected from the group consisting of acetic acid vinyl.maleic acid anhydride copolymer, styrene.maleic acid anhydride copolymer, styrene.maleic acid monoester copolymer, vinylmethylether.maleic acid anhydride copolymer, ethylene.maleic acid anhydride copolymer, vinylbutylether.maleic acid anhydride copolymer, acrylonitrile.acrylic acid methyl.maleic acid anhydride copolymer and acrylic acid butyl.styrene.maleic acid anhydride copolymer; enteric polyvinyl derivative selected from the group consisting of polyvinylalcohol phthalate, polyvinylacetal phthalate, polyvinylbutyrate phthalate and polyvinylacetoacetal phthalate; and a mixture thereof.

Besides the aforementioned polymers and active ingredients, a tablet layer herein may further comprise pharmaceutically acceptable fillers such as starch, microcrystalline cellulose, lactose, glucose, mannitol, alginate, salt of alkaline earth metal, clay, polyethylene glycol and dicalcium phosphate in such an amount that may not lower the effect of the present invention.

Examples of the binder include starch, microcrystalline cellulose, highly-dispersed silica, mannitol, lactose, polyethylene glycol, polyvinylpyrrolidone, hydroxypropyl methyl cellulose, hydroxypropyl cellulose, natural gum, synthetic gum, copovidone and gelatin.

Examples of the disintegrant as the aforementioned additives include starch or denatured starch such as sodium starch glycolate, corn starch, potato starch and pre-gelatinized starch; clay such as bentonite, montmorillonite and veegum; celluloses such as microcrystalline cellulose, hydroxypropyl cellulose and carboxymethyl cellulose; aligns such as sodium alginate or alginic acid; crosslinked celluloses such as croscarmellose sodium; gums such as guar gum and xanthan gum; a crosslinked polymer such as crospovidone; and effervescent formulation such as sodium bicarbonate and citric acid.

Examples of the lubricant include talc, magnesium stearate and alkaline earth metal stearate type calcium, zinc, etc, sodium lauryl sulfate, hydrogenated vegetable oil, sodium benzoate, sodium stearyl fumarate, glyceryl monostearate and polyethylene glycol. Other pharmaceutically acceptable additives such as coloring agents or perfumery may be used.

Additives that may be used in the present invention are not limited to the aforementioned ones, and the amount of the additives may be determined in a conventional manner.

A combination preparation herein may also be formulated so as to comprise a coating layer on the aforementioned tablet layer. The coating layer may contain a film former, a film-forming adjuvant or a mixture thereof.

The physical partition mentioned above may be embodied into various formulations, such as for example tablets, powders, granules and capsules that comprise an uncoated tablet, a coated tablet, a multi-layered tablet or delayed release core tablet in a tablet.

In a tablet herein, the dihydropyridine-based calcium channel blocker may be contained in the amount of 1-60 mg, preferably 2.5-30 mg, and the ARB may be contained in the amount of 1-600 mg, preferably 2.5-200 mg.

Hereunder is provided a detailed description for the manufacturing process of a combination preparation comprising a dihydropyridine-based calcium channel blocker and an ARB according to the present invention.

In the first step, a delayed-immediate-release granule or coated tablet is prepared by mixing or granulating or coating a dihydropyridine-based calcium channel blocker with a release-controlling material selected from the group consisting of water-soluble polymer, water-insoluble polymer, enteric polymer and a mixture thereof together with pharmaceutically acceptable additives, followed by granulation and coating.

In the second step, an immediate-release particle or granule including ARB with pharmaceutically acceptable additives is prepared by performing normal processes for oral solid forms, such as kneading, drying and sieving.

In the third step, a dosage form for oral administration is prepared by compression or filling after mixing the granules or coated tablets prepared in the first and the second steps with pharmaceutically acceptable excipient, followed by compression or filling.

Hereunder is provided a detailed description of each step above.

A. Preparation of Tablets

Tablets of uniform weight are prepared by mixing the particles or granules prepared in the first step, which may be optionally coated with a release-controlling material, with the granules prepared in the second step, followed by compression. The resulting tablets may be needed film-coating for improving stability or shape.

B. Preparation of Multi-Layered Tablets

The granules prepared in the first step are optionally coated with a release-controlling material, and dried. The dried granules are compressed with the granules prepared in the second step by using a multi-layered tablet press, thereby obtaining double-layered tablet. Optoinally, triple or more of multi-layered tablet may also be prepared by further adding a release adjuvant layer on the double-layered tablet. Coated multi-layered tablet may be prepared by coating the multi-layered tablet.

C. Preparation of Inner Core Tablets

The coated tablets or granules prepared in the first step are optionally coated with a release-controlling material and dried, followed by the compression into uniform weight. The resulting granules are used as an inner core optionally after performing further coating, and compressed with the granules prepared in the second step by using an inner core tablet press, thereby providing inner core tablets. Coated inner core tablets may be prepared by coating the inner core tablets.

D. Preparation of Capsules (Granules)

The granules prepared in the first step may be optionally coated with a release-controlling material, and dried. The dried granules are filled in prepared in the second step with the granules prepared in the second step, and the amount of each granule may be determined considering the effective amount of each ingredient.

E. Preparation of Capsules (Pellets)

A dihydropyridine-based calcium channel blocker and a release-controlling material or pharmaceutically acceptable additives are dissolved or suspended in water or organic solvent or a mixed solvent. This solution or suspension is coated on sugar spheres and dried. This is mixed with the granules prepared in the second step and filled in a capsule, thereby provide capsules. Optionally, a release-controlling solution may also be filled in this capsule. The release-controlling solution is prepared by dissolving a release-controlling material or a combination thereof in water or organic solvent or a mixed solvent and dried.

Once-daily administration of those prepared combination preparation herein especially in the evening allows the constant activities in controlling hypertension and in preventing its complications.

Appropriate dosage of a combination preparation herein may be determined considering rates of absorption and excretion, degree of inactivation and age, sex and health conditions of a patient. Preferably, an appropriate daily dosage for an adult is 6.5-350 mg, more preferably 2.5-40.0 mg of dihydropyridine-based calcium channel blocker and 2.5-300 mg of ARB.

A combination preparation is preferred to be administered in the evening for the following reasons.

Like other drugs, amlodipine and losartan show a characteristic biorhythmic of activity during 24 hours [J. Clin. Hypertens 5(1): 17-23, 30, 2003].

That is, amlodipine shows the highest activity of inhibiting the increase in blood pressure from 1 a.m. to 1 p.m. This is because spasmodic phenomenon in wall of blood vessel may be severe during the day time due to various stress factors and amlodipine directly acts on the vasmospasmodic increase in blood pressure.

Thus, when administered early in the evening around 7 p.m., a functional combination preparation herein may maintain the appropriate level of blood pressure during the day time because amlodipine reaches the maximum plasma concentration after 4 a.m.

Meanwhile, losartan is usually weaker than amlodipine in the activity of lowering blood pressure. During 4-12 p.m., however, losartan is more active because losartan inhibit and suppress the generation of aldosterone and the activity of angiotensin-2 that are generated and act usually at night. This means that the elevation of blood pressure at night may be prevented effectively.

In particular, it is expected that a combination preparation herein may be very useful for a patient suffering from complications of hypertension, which requires the maintenance of a constant level of blood pressure and the inhibition of sympathetic overexcitation in heart for 24 hours.

Table 3 shows the comparison between a functional combination preparation herein and the conventional co-administration prescription method. It was ascertained that a functional combination preparation according to the present invention is superior to the conventional co-administration method in the treatment of hypertension. Further, a functional combination preparation according to the present invention will decrease side effect caused by the interaction between drugs and will reduce the frequency of complications by acting more actively during the time when the risk of complications is relatively higher. Further, as all the effects of the present invention may be achieved by simple medication, i.e., once-daily administration in the evening, it is expected that the present invention facilitates a doctor's prescription and medication instruction and improves medication compliance, thereby reducing the frequency of medical accident and the time for a doctors' prescription and medication instruction.

TABLE 3
Comparison between a functional combination preparation
herein and the conventional co-administration method

| | The conventional co- | | A functional combination |
|---|---|---|---|
| | administration method | | preparation herein |
| Administration time | Usually in the morning | | In the evening |
| Release and | Simultaneously released and | | Chronotherapeutically |
| absorption of two | absorbed in the morning | | released and absorbed in the |
| ingredients | | | evening |
| Time of more activity | 10 a.m.-10 p.m. | | 10 p.m.-10 a.m. |
| in controlling | | | |
| hypertension | | | |
| For controlling non- | Inappropriate | | Effective in non-dipper |
| dipper hypertension | | | hypertension where the risk of |
| | | | complications is high |
| Preventive activity | (1) | It is difficult to maintain | Effective in non-dipper |
| during the risky time | | proper plasma pressure at | hypertension patients who |
| of complications | | risky time if administered | have higher risk rate of |
| (from dawn to | | after the morning meal. | complications |
| morning) | (2) | Although administered in | |
| | | the evening, the activity of | |
| | | inhibiting the increase in | |
| | | blood pressure is reduced | |
| | | because the two | |
| | | ingredients are | |
| | | simultaneously released | |
| | | and then antagonize with | |
| | | each other in the liver | |
| | | enzyme. | |

| | | |
|---|---|---|
| Interaction between | The two ingredients are | There is no antagonism |
| two ingredients | simultaneously released and | between the two ingredients. |
| | antagonize with each other in | Losartan is first absorbed |
| | the liver by the action of the | from the intestine and |
| | same enzyme (CYP3A4). The | activated by the catalyst in the |
| | activity of losartan is reduced | liver(CYP3A4). And then |
| | because amlodipine inhibits | amlodipine passes through |
| | activity of the enzyme | the liver 3-4 hours later. |
| | (CYP3A4) that activates the | |
| | losartan. | |

As aforementioned, a chronotherapeutic administration herein is superior to a co-administration and that a nighttime administration is superior to a daytime administration in anti-hypertensive activity.

### Mode for Invention

The present invention is described more specifically by the following Examples. Examples herein are meant only to illustrate the present invention, but in no way to limit the claimed invention.

### Example 1

### Preparation of Single Pills

(1) Preparation of Granules of Losartan Potassium

Predetermined amounts of losartan potassium, lactose and microcrystalline cellulose as shown in Table 4 were sieved with a No. 35 sieve, and mixed using a double cone mixer for 5 minutes. The mixture was placed into a fluidized-bed granulator (GPCG 1: Glatt), and sprayed with a binder solution (an aqueous solution of hydroxypropylmethyl cellulose) to prepare granules, and dried. The granules were added with carbomer 71G powders, and mixed with magnesium stearate with a double cone mixer. The resulting mixture was compressed using a rotary tablet press (MRC-33: Sejong) at a speed of 30 turns per minute to provide tablets with a hardness of 7-9 kp, a thickness of 3.0 mm and a diameter of 5.5 mm.

Predetermined amounts of losartan potassium, lactose and microcrystalline cellulose as shown in Table 4 were sieved with a No. 35 sieve, and mixed in a double cone mixer for 5 minutes. A binder solution was prepared by dissolving hydroxypropylcellulose in purified water. The mixture was placed in a fluidized-bed granulator, and granulated by adding a binder solution. A high-speed mixer may be used during the granulation process. GPCG-1 (Glatt, Germany) was used as a fluidized-bed granulator, and operated using a top-spray system. After adding the mixture, the granulator was preheated under the following conditions: air flow of 80 m³/h and inlet air temperature of 40° C., and filter shaking (delta P filter was maintained <500 pa) was conducted in the asynchronous mode for 40 seconds. The binder solution was sprayed at a speed of 1.0-10 g/minute for granulation when the temperature reached 35° C., and the spraying angle of the coating solution was controlled, while maintaining the pressure of the atomizing air within 1.0-2.0 bar. Particles began to be formed as the process proceeded, and the air flow was increased from 80 m³/h to 120 m³/h. Filter shaking (delta P filter was maintained <4000 pa) was conducted in the synchronous mode for 5 seconds in a minute to prevent the loss of particles.

The granulated particles were dried in a fluidized-bed dryer by using GPCG-1 (Glatt, Germany) as a fluidized-bed granule dryer under the following conditions: air flow of 120 m³/h and inlet air temperature of 65° C. Filter shaking (delta P filter was maintained <4000 pa) was conducted in the asynchronous mode for 5 seconds in 30 seconds. When the temperature reached 40° C., samples were measured. The drying process was completed when the loss of drying (LOD) was lower than (on drying(LOD) less than) 2.5%, while the particles were further dried when the standard was not satisfied. The dried mixture was sieved with an F-type grinder equipped with a No. 20 sieve. This was placed into a double-cone mixer and mixed with pregelatinized starch for 10 minutes. Immediate-release granules comprising losartan were prepared by adding magnesium stearate, followed by mixing for 4 minutes.

(2) Preparation of Delayed-Immediate-Release Granules of Amlodipine

Predetermined amounts of amlodipine maleate, microcrystalline cellulose, crosslinked polyvinylpyrrolidone and sodium chloride as shown in Table 4 were sieved with a No. 35 sieve, and mixed using a double cone mixer for 5 minutes. A binder solution was prepared by dissolving hydroxypropylcellulose in purified water. The fluidized-bed granulation and the fluidized-bed drying were conducted under the same conditions as in the process of preparing intermediate-release granules of losartan. The dried mixture was placed in a fluidized-bed granule coating machine, and coated with a solution prepared by dissolving cellulose acetate (acetal group 32%), cellulose acetate (acetal group 39.8%) and hydroxypropylmethyl cellulose in a mixture of ethanol and methylene chloride.

The coating process was conducted with GPCG-1 (Glatt, Germany) using a bottom-spray system. B-type or C-type plate was used depending on the size of granules. A partition gap was at 25 mm position and a spray nozzle of 1 mm size was equipped. The machine is preheated under the following conditions: air flow of 100 m³/h, inlet air temperature of 45-60° C. and particle temperature of 40-50° C., and filter shaking (delta P filter was maintained <500 pa) was conducted in the asynchronous mode for 5 seconds in 30 seconds.

Film coating solution was sprayed at a speed of 1-5 g/minute when the particle temperature reached 35° C. during the pretreatment process, and the spraying angle of the coating solution was controlled, while maintaining the pressure of the atomizing air within 1.0-1.5 bar. The temperature of particles was maintained within 34-38° C. during the process, and drying and surface treatment were conducted for about an hour while maintaining the goods temperature at 40 ® C. Delayed-immediate-release granules of amlodipine were prepared by mixing the particles with magnesium stearate for 4 minutes.

The two kinds of granules were mixed and compressed with a rotary tablet press (MRC-33, Sejong Mechanics, Korea) equipped with a punch of diameter 10.0 mm. The resulting tablet was coated with a solution prepared by dissolving hydroxypropylmethylcellulose 2910, polyethyleneglycol 6,000 and titanium oxide in a mixture of ethanol and methylene chloride under the conventional conditions.

### Example 2

As shown in Table 4, tablets were prepared the same as in Example 1 except by using only cellulose acetate (acetal group 32%) instead of using cellulose acetate (acetal group 32%), cellulose acetate (acetal group 39.8%) and hydroxypropylmethylcellulose.

### Example 3

As shown in Table 4, tablets were prepared the same as in Example 1 except by using cellulose acetate (acetal group 32%), cellulose acetate (acetal group 39.8%) and hydroxypropylmethylcellulose as shown in Table 4.

### Example 4

As shown in Table 4, tablets were prepared the same as in Example 1 except that a solution was prepared by dissolving ethylcellulose in a mixture of ethanol, methylene chloride and that the solution was further coated on the granules coated with cellulose acetate, followed by the addition of magnesium stearate before mixing for 4 minutes.

### Example 5

As shown in Table 4, tablets were prepared the same as in Example 4 except by replacing ethylcellulose with Eudragit RL.

### Example 6

As shown in Table 4, tablets were prepared the same as in Example 4 except by replacing ethylcellulose with Eudragit RS.

### Example 7

As shown in Table 4, tablets were prepared the same as in Example 4 except by using ethylcellulose and hydroxypropylmethylcellulose phthalate instead of ethylcellulose.

### Example 8

### Preparation of Multi-Layered Tablets

The amlodipine delayed-immediate-release granules prepared in Example 4 and the losartan immediate-release granules prepared in Example 1 were separately introduced into each different inlet of multi-layered tablet press(MRC-37T: Sejong Mechanics, Korea) equipped with a punch of diameter 10 mm. The compressed tablets were coated with a solution prepared by dissolving hydroxypropylmethylcellulose 2910, polyethyleneglycol 6,000 and titanium oxide in a mixture of ethanol and methylene chloride under the conventional conditions.

### Example 9

### Preparation of Inner Core Tablets

The amlodipine delayed-immediate-release granules prepared in Example 4 were compressed in a rotary tablet press (RUD-I: kilian, Germany) equipped with a punch of diameter 8 mm, thus obtaining inner core tablets. The inner core tablets were compressed together with the losartan immediate-release granules prepared in Example 1 in an inner core tablet press equipped with a punch of diameter 10 mm. The resulting tablets were coated with a solution prepared by dissolving hydroxypropylmethylcellulose 2910, polyethyleneglycol 6,000 and titanium oxide in a mixture of ethanol and methylene chloride under the conventional conditions.

### Example 10

### Preparation of Capsules

The amlodipine delayed-immediate-release granules prepared in Example 4 were mixed with the losartan immediate-release granules prepared in Example 1, and filled in a capsule (No. 0 or 1) by using a capsule filler, thereby providing capsules.

### Example 11

### Preparation of Amlodipine-Valsartan Multi-Layered Tablets

As shown in Table 4, tablets were prepared the same as in Example 8 except by using valsartan and calcium phosphate instead of losartan potassium and lactose.

### Example 12

### Preparation of Amlodipine-Telmisartan Multi-Layered Tablets

As shown in Table 4, tablets were prepared the same as in Example 8 except by using sodium hydroxide and telmisartan instead of losartan potassium and lactose.

### Example 13

### Preparation of Amlodipine-Candesartan Multi-Layered Tablets

As shown in Table 4, tablets were prepared the same as in Example 8 except by using candesartan cilexetil instead of losartan potassium.

### Example 14

### Preparation of Amlodipine-Irbesartan Multi-Layered Tablets

As shown in Table 5, tablets were prepared the same as in Example 8 except by using irbesartan instead of losartan potassium.

### Example 15

### Preparation of Amlodipine-Olmesartan Multi-Layered Tablets

As shown in Table 5, tablets were prepared the same as in Example 8 except by using olmesartan medoxomil instead of losartan.

### Example 16

### Preparation of Lercanidipine-Losartan Multi-Layered Tablets

As shown in Table 5, tablets were prepared the same as in Example 8 except by using lercanidipine hydrochloride instead of amlodipine.

### Example 17

### Preparation of Lacidipine-Losartan Multi-Layered Tablets

As shown in Table 5, tablets were prepared the same as in Example 8 except by using lacidipine instead of amlodipine.

### Example 18

### Preparation of Amlodipine-Losartan Inner Core Tablets

(1) Preparation of Amlodipine Delayed-Immediate-Release Layer

As shown in Table 5, amlodipine besylate and microcrystalline cellulose were sieved with a No. 35 sieve and mixed in a double cone mixer. This mixture was introduced into a fluidized-bed granulator (GPCG 1: Glatt). The mixture were introduced into a fluidized-bed granulator (GPCG 1: Glatt), granulated by spraying a binder solution (an aqueous solution of hydroxypropylmethyl cellulose) and dried. The granules were mixed with carbomer 71G powders for 10 minutes, and added with magnesium stearate in a double cone mixer. The resulting mixture was compressed using a rotary tablet press (MRC-33: Sejong) to provide tablets with a diameter of 5.5 mm. These tablets were coated with hydroxypropylmethylcellulose phthalate, and used as inner core tablets.

(2) Preparation of Losartan Immediate-Release Layer Granules

As shown in Table 5, losartan, microcrystalline cellulose, lactose and pregelatinized starch were sieved with a No. 35 sieve, and mixed in a double cone mixer for 20 minutes. Magnesium stearate was also sieved with a No. 35 sieve, and introduced into the double cone mixer, and mixed for 4 minutes to provide immediate-release granules having a losartan layer.

(3) Post-Mixing, Compression and Coating

Inner core tablets were prepared by compressing amlodipine inner core tablets and a composition containing losartan (outer layer) in an inner core tablet press (RUD-1: Kilian), followed by the formation of film coating layer using Hi-coater (SFC-30N, Sejong Mechanics, Korea).

### Example 19

### Preparation of Amlodipine—Losartan Inner Core Tablets

(1) Preparation of Amlodipine Delayed-Immediate-Release Layer

As shown in Table 5, amlodipine besylate and microcrystalline cellulose were sieved with a 35 mesh sieve, and mixed in a double cone mixer. The mixture were introduced into a fluidized-bed granulator (GPCG 1: Glatt), granulated by spraying a binder solution (an aqueous solution of hydroxypropylmethyl cellulose) and dried. The granules were mixed with magnesium stearate in a double cone mixer, and compressed using a rotary tablet press (MRC-33: Sejong) to provide tablets with a diameter of 5.5 mm. These tablets were coated with ethylcellulose, and used as inner core tablets.

(2) Preparation of Losartan Immediate-Release Layer Granules

As shown in Table 5, losartan, microcrystalline cellulose, lactose and pregelatinized starch were sieved with a No. 35 sieve, and mixed in a double cone mixer for 20 minutes. Magnesium stearate was also sieved with a No. 35 sieve, and introduced into the double cone mixer, and mixed for 4 minutes to provide immediate-release granules having a losartan layer.

(3) Post-Mixing, Compression and Coating

Inner core tablets were prepared by compressing amlodipine inner core tablets and a composition containing losartan (outer layer) in an inner core tablet press (RUD-1: Kilian), followed by the formation of film coating layer using Hi-coater (SFC-30N, Sejong Mechanics, Korea).

### Example 20

### Preparation of Amlodipine-Valsartan Inner Core Tablets

As shown in Table 5, tablets were prepared the same as in Example 18 except by using valsartan and lactose instead of losartan potassium and calcium phosphate.

### Example 21

### Preparation of Amlodipine-Telmisartan Inner Core Tablets

As shown in Table 5, tablets were prepared the same as in Example 18 except by using telmisartan and sodium hydroxide instead of losartan potassium and lactose.

### Example 22

### Preparation of Amlodipine-Candesartan Inner Core Tablets

As shown in Table 5, tablets were prepared the same as in Example 18 except by using candesartan cilexetil instead of losartan potassium.

### Example 23

### Preparation of Amlodipine-Irbesartan Inner Core Tablets

As shown in Table 5, tablets were prepared the same as in Example 18 except by using irbesartan instead of losartan potassium.

### Example 24

### Preparation of Amlodipine-Olmesartan Inner Core Tablets

As shown in Table 5, tablets were prepared the same as in Example 18 except by using olmesartan medoxomil instead of losartan.

### Example 25

### Preparation of Lercanidipine-Losartan Inner Core Tablets

As shown in Table 5, tablets were prepared the same as in Example 18 except by using lercanidipine hydrochloride instead of amlodipine.

### Example 26

### Preparation of Lacidipine-Losartan Inner Core Tablets

As shown in Table 5, tablets were prepared the same as in Example 18 except by using lacidipine instead of amlodipine.

TABLE 4

| | | Contents (mg/tablet) | | | | | | | | | | | | |
|---|---|---|---|---|---|---|---|---|---|---|---|---|---|---|
| Ingredients | | Ex. 1 | Ex. 2 | Ex. 3 | Ex. 4 | Ex. 5 | Ex. 6 | Ex. 7 | Ex. 8 | Ex. 9 | Ex. 10 | Ex. 11 | Ex. 12 | Ex. 13 |
| ARB drugs | Losartan | 50.0 | 50.0 | 50.0 | 50.0 | 50.0 | 50.0 | 50.0 | 50.0 | 50.0 | 50.0 | — | — | — |
| | potassium | | | | | | | | | | | | | |
| | Valsartan | — | — | — | — | — | — | — | — | — | — | 80.0 | — | — |
| | Telmisartan | — | — | — | — | — | — | — | — | — | — | — | 40.0 | — |
| | Candesartancilexetil | — | — | — | — | — | — | — | — | — | — | — | — | 16.0 |
| | Irbesartan | — | — | — | — | — | — | — | — | — | — | — | — | — |
| | Olmesartan | — | — | — | — | — | — | — | — | — | — | — | — | — |
| | medoxomil | | | | | | | | | | | | | |
| Filler | Lactose | 55.0 | 55.0 | 55.0 | 55.0 | 55.0 | 55.0 | 55.0 | 55.0 | 55.0 | 55.0 | — | — | 55.0 |
| Filler | Microcrystalline | 50.0 | 50.0 | 50.0 | 50.0 | 50.0 | 50.0 | 50.0 | 50.0 | 50.0 | 50.0 | 50.0 | 50.0 | 50.0 |
| | cellulose | | | | | | | | | | | | | |
| Alkalizer | Sodium | — | — | — | — | — | — | — | — | — | — | — | 55 | — |
| | hydroxide | | | | | | | | | | | | | |
| Filler | Calcium | — | — | — | — | — | — | — | — | — | — | 55 | — | — |
| | phosphate | | | | | | | | | | | | | |
| Binder | Hydroxypropyl | 3.0 | 3.0 | 3.0 | 3.0 | 3.0 | 3.0 | 3.0 | 3.0 | 3.0 | 3.0 | 3.0 | 3.0 | 3.0 |
| | cellulose | | | | | | | | | | | | | |
| Lubricant | Magnesium | 2.0 | 2.0 | 2.0 | 2.0 | 2.0 | 2.0 | 2.0 | 2.0 | 2.0 | 2.0 | 2.0 | 2.0 | 2.0 |
| | stearate | | | | | | | | | | | | | |
| Solvent | Purified | 10.0 | 10.0 | 10.0 | 10.0 | 10.0 | 10.0 | 10.0 | 10.0 | 10.0 | 10.0 | 10.0 | 10.0 | 10.0 |
| | water | | | | | | | | | | | | | |
| | (volatile) | | | | | | | | | | | | | |
| Total | | 160.0 | 160.0 | 160.0 | 160.0 | 160.0 | 160.0 | 160.0 | 160.0 | 160.0 | 160.0 | 190.0 | 150.0 | 126.0 |
| Calcium | Amlodipine | 6.4 | 6.4 | 6.4 | 6.4 | 6.4 | 6.4 | 6.4 | 6.4 | 6.4 | 6.4 | 6.4 | 6.4 | 6.4 |
| antagonist | maleate | | | | | | | | | | | | | |
| | Lercanidipine | — | — | — | — | — | — | — | — | — | — | — | — | — |
| | hydrochloride | | | | | | | | | | | | | |
| Filler | Microcrystalline | 325.6 | 325.6 | 325.6 | 325.6 | 325.6 | 325.6 | 325.6 | 325.6 | 325.6 | 325.6 | 325.6 | 325.6 | 325.6 |
| | cellulose | | | | | | | | | | | | | |
| Disintegrant | Crosslinked | 50.0 | 50.0 | 50.0 | 50.0 | 50.0 | 50.0 | 50.0 | 50.0 | 50.0 | 50.0 | 50.0 | 50.0 | 50.0 |
| | polyvinylpyrrolidone | | | | | | | | | | | | | |
| Osmotic | Chloride | 25.0 | 25.0 | 25.0 | 25.0 | 25.0 | 25.0 | 25.0 | 25.0 | 25.0 | 25.0 | 25.0 | 25.0 | 25.0 |
| agent | sodium | | | | | | | | | | | | | |
| Binder | Hydroxypropyl | 5.0 | 5.0 | 5.0 | 5.0 | 5.0 | 5.0 | 5.0 | 5.0 | 5.0 | 5.0 | 5.0 | 5.0 | 5.0 |
| | cellulose | | | | | | | | | | | | | |
| Semipermeable | Cellulose | 20.0 | 42.0 | — | 20.0 | 20.0 | 20.0 | 20.0 | 20.0 | 20.0 | 20.0 | 20.0 | 20.0 | 20.0 |
| polymer | acetate 320S | | | | | | | | | | | | | |
| | Cellulose | 20.0 | — | — | 20.0 | 20.0 | 20.0 | 20.0 | 20.0 | 20.0 | 20.0 | 20.0 | 20.0 | 20.0 |
| | acetate | | | | | | | | | | | | | |
| | 398NF10 | | | | | | | | | | | | | |
| Water- | Ethylcellulose | — | — | 42.0 | 10.0 | — | — | 5.0 | 10.0 | 10.0 | 10.0 | 10.0 | 10.0 | 10.0 |
| insoluble | | | | | | | | | | | | | | |
| polymer | | | | | | | | | | | | | | |
| Water- | Hydroxypropyl- | 2.0 | — | — | 2.0 | 2.0 | 2.0 | 2.0 | 2.0 | 2.0 | 2.0 | 2.0 | 2.0 | 2.0 |
| soluble | methylcellulose | | | | | | | | | | | | | |
| polymer | | | | | | | | | | | | | | |
| Sustained- | Eudragit RL | — | — | — | — | 10.0 | — | — | — | — | — | — | — | — |
| release | Eudragit RS | — | — | — | — | — | 10.0 | — | — | — | — | — | — | — |
| excipient | | | | | | | | | | | | | | |
| Enteric | Hydroxypropyl- | — | — | — | — | — | — | 10.0 | — | — | — | — | — | — |
| polymer | methylcellulose | | | | | | | | | | | | | |
| | phthalate | | | | | | | | | | | | | |
| Lubricant | Magnesium | 1.0 | 1.0 | 1.0 | 1.0 | 1.0 | 1.0 | 1.0 | 1.0 | 1.0 | 1.0 | 1.0 | 1.0 | 1.0 |
| | stearate | | | | | | | | | | | | | |
| Solvent | Purified | 15.0 | 15.0 | 15.0 | 15.0 | 15.0 | 15.0 | 15.0 | 15.0 | 15.0 | 15.0 | 15.0 | 15.0 | 15.0 |
| | water | | | | | | | | | | | | | |
| | (volatile) | | | | | | | | | | | | | |
| Solvent | Ethanol | 200.0 | 200.0 | 200.0 | 200.0 | 200.0 | 200.0 | 200.0 | 200.0 | 200.0 | 200.0 | 200.0 | 200.0 | 200.0 |
| | (volatile) | | | | | | | | | | | | | |
| Solvent | Methylene | 200.0 | 200.0 | 200.0 | 200.0 | 200.0 | 200.0 | 200.0 | 200.0 | 200.0 | 200.0 | 200.0 | 200.0 | 200.0 |
| | chloride | | | | | | | | | | | | | |
| | (volatile) | | | | | | | | | | | | | |
| Total | | 155.0 | 155.0 | 155.0 | 165.0 | 165.0 | 165.0 | 170.0 | 165.0 | 165.0 | 165.0 | 165.0 | 165.0 | 165.0 |
| Film coating | Hydroxypropyl- | 9.0 | 9.0 | 9.0 | 9.0 | 9.0 | 9.0 | 9.0 | 9.0 | 9.0 | — | 9.0 | 9.0 | 9.0 |
| agent | methylcellulose | | | | | | | | | | | | | |
| | 2910 | | | | | | | | | | | | | |
| Plasticizer | Polyethyleneglycol | 0.7 | 0.7 | 0.7 | 0.7 | 0.7 | 0.7 | 0.7 | 0.7 | 0.7 | — | 0.7 | 0.7 | 0.7 |
| | 6000 | | | | | | | | | | | | | |
| Coloring | Titanium | 0.3 | 0.3 | 0.3 | 0.3 | 0.3 | 0.3 | 0.3 | 0.3 | 0.3 | — | 0.3 | 0.3 | 0.3 |
| agent | oxide | | | | | | | | | | | | | |
| Film coating | Ethanol | 100.0 | 100.0 | 100.0 | 100.0 | 100.0 | 100.0 | 100.0 | 100.0 | 100.0 | — | 100.0 | 100.0 | 100.0 |
| solvent | (volatile) | | | | | | | | | | | | | |
| Film coating | Methylene | 100.0 | 100.0 | 100.0 | 100.0 | 100.0 | 100.0 | 100.0 | 100.0 | 100.0 | — | 100.0 | 100.0 | 100.0 |
| solvent | chloride | | | | | | | | | | | | | |
| | (volatile) | | | | | | | | | | | | | |

| | | | | | | | | | | | | | |
|---|---|---|---|---|---|---|---|---|---|---|---|---|---|
| Total | 325.0 | 325.0 | 325.0 | 335.0 | 335.0 | 335.0 | 335.0 | 335.0 | 335.0 | 325.0 | 365.0 | 325.0 | 301.0 |
| Formulation for oral | Tablet | Tablet | Tab- | Tab- | Tab- | Tab- | Tab- | Multi- | Inner | Capsule | Multi- | Multi- | Multi- |
| administration | | | let | let | let | let | let | layered | core | | layered | layered | layered |
| | | | | | | | | tablet | tablet | | tablet | tablet | tablet |

TABLE 5

| | | Contents (mg/tablet) | | | | | | | | | | | | |
|---|---|---|---|---|---|---|---|---|---|---|---|---|---|---|
| | | | | | | | | | | | | | | Ex. |
| Ingredients | | Ex. 14 | Ex. 15 | Ex. 16 | Ex. 17 | Ex. 18 | Ex. 19 | Ex. 20 | Ex. 21 | Ex. 22 | Ex. 23 | Ex. 24 | Ex. 25 | 26 |
| ARB | Losartan | — | — | 50.0 | 50.0 | 50.0 | 50.0 | — | — | — | — | — | 50.0 | 50.0 |
| drugs | potassium | | | | | | | | | | | | | |
| | Valsartan | — | — | — | — | — | — | 80.0 | — | — | — | — | — | — |
| | Telmisartan | — | — | — | — | — | — | — | 40.0 | — | — | — | — | — |
| | Candesartan- | — | — | — | — | — | — | — | — | 16.0 | — | — | — | — |
| | cilexetil | | | | | | | | | | | | | |
| | Irbesartan | 150 | — | — | — | — | — | — | — | — | 150.0 | — | — | — |
| | Olmesartan | — | 20.0 | — | — | — | — | — | — | — | — | 20.0 | — | — |
| | medoxomil | | | | | | | | | | | | | |
| Filler | Lactose | 55.0 | 55.0 | 55.0 | 55.0 | 100.0 | 100.0 | 100.0 | 100.0 | 100.0 | 100.0 | 100.0 | 100.0 | 100.0 |
| Filler | Micro- | 50.0 | 50.0 | 50.0 | 50.0 | 175.0 | 175.0 | 175.0 | 175.0 | 175.0 | 175.0 | 175.0 | 175.0 | 175.0 |
| | crystalline | | | | | | | | | | | | | |
| | cellulose | | | | | | | | | | | | | |
| Filler | Pregelatinized | — | — | — | — | 65.0 | 65.0 | 65.0 | 65.0 | 65.0 | 65.0 | 65.0 | 65.0 | 65.0 |
| | starch | | | | | | | | | | | | | |
| Binder | Hydroxypropyl | 3.0 | 3.0 | 3.0 | 3.0 | — | — | — | — | — | — | — | — | — |
| | cellulose | | | | | | | | | | | | | |
| Lubricant | Magnesium | 2.0 | 2.0 | 2.0 | 2.0 | 5.0 | 5.0 | 5.0 | 5.0 | 5.0 | 5.0 | 5.0 | 5.0 | 5.0 |
| | stearate | | | | | | | | | | | | | |
| Solvent | Purified | 10.0 | 10.0 | 10.0 | 10.0 | — | — | — | — | — | — | — | — | — |
| | water | | | | | | | | | | | | | |
| | (volatile) | | | | | | | | | | | | | |
| Total | | 260.0 | 130.0 | 160.0 | 160.0 | 395.0 | 395.0 | 425.0 | 385.0 | 361.0 | 495.0 | 365.0 | 395.0 | 395.0 |
| Calcium | Amlodipine | 6.4 | 6.4 | — | — | — | — | — | — | — | — | — | — | — |
| antagonist | maleate | | | | | | | | | | | | | |
| | Amlodipine | — | — | — | — | 6.94 | 6.94 | 6.94 | 6.94 | 6.94 | 6.94 | 6.94 | — | — |
| | besylate | | | | | | | | | | | | | |
| | Lercanidipine | — | — | 10.0 | — | — | — | — | — | — | — | — | 10.0 | — |
| | hydrochloride | | | | | | | | | | | | | |
| | Lacidipine | — | — | — | 4.0 | — | — | — | — | — | — | — | — | 4.0 |
| Filler | Micro- | 25.6 | 25.6 | 22.0 | 27.0 | 80.06 | 80.06 | 80.06 | 80.06 | 80.06 | 80.06 | 80.06 | 80.0 | 80.0 |
| | crystalline | | | | | | | | | | | | | |
| | cellulose | | | | | | | | | | | | | |
| Dis- | Crosslinked | 50.0 | 50.0 | 50.0 | 50.0 | — | — | — | — | — | — | — | — | — |
| integrant | polyvinyl- | | | | | | | | | | | | | |
| | pyrrolidone | | | | | | | | | | | | | |
| Osmotic | Sodium | 25.0 | 25.0 | 25.0 | 25.0 | — | — | — | — | — | — | — | — | — |
| agent | Chloride | | | | | | | | | | | | | |
| Binder | Hydroxypropyl | 5.0 | 5.0 | 5.0 | 5.0 | — | — | — | — | — | — | — | — | — |
| | cellulose | | | | | | | | | | | | | |
| Semi- | Cellulose | 20.0 | 20.0 | 20.0 | 20.0 | — | — | — | — | — | — | — | — | — |
| permeable | acetate 320S | | | | | | | | | | | | | |
| polymer | Cellulose | 20.0 | 20.0 | 20.0 | 20.0 | — | — | — | — | — | — | — | — | — |
| | acetate | | | | | | | | | | | | | |
| | 398NF10 | | | | | | | | | | | | | |
| Water- | Ethylcellulose | 10.0 | 10.0 | 10.0 | 10.0 | — | 10.0 | — | — | — | — | — | — | — |
| insoluble | | | | | | | | | | | | | | |
| polymer | | | | | | | | | | | | | | |
| Water- | Carbomer | — | — | — | — | 10.0 | — | — | 10.0 | 10.0 | 10.0 | 10.0 | 10.0 | 10.0 |
| insoluble | 71G | | | | | | | | | | | | | |
| polymer | | | | | | | | | | | | | | |
| Water- | Hydroxy- | 2.0 | 2.0 | 2.0 | 2.0 | 2.0 | 2.0 | 2.0 | 2.0 | 2.0 | 2.0 | 2.0 | 2.0 | 2.0 |
| soluble | propyl- | | | | | | | | | | | | | |
| polymer | methylcellulose | | | | | | | | | | | | | |
| Sustained- | Eudragit RL | — | — | — | — | — | — | — | — | — | — | — | — | — |
| release | Eudragit RS | — | — | — | — | — | — | — | — | — | — | — | — | — |
| excipient | | | | | | | | | | | | | | |
| Enteric | Hydroxy- | — | — | — | — | 20.0 | — | 20.0 | 20.0 | 20.0 | 20.0 | 20.0 | 20.0 | 20.0 |
| polymer | propyl- | | | | | | | | | | | | | |
| | methylcellulose | | | | | | | | | | | | | |
| | phthalate | | | | | | | | | | | | | |
| Lubricant | Magnesium | 1.0 | 1.0 | 1.0 | 1.0 | 1.0 | 1.0 | 1.0 | 1.0 | 1.0 | 1.0 | 1.0 | 1.0 | 1.0 |
| | stearate | | | | | | | | | | | | | |
| Solvent | Purified | 15.0 | 15.0 | 15.0 | 15.0 | 15.0 | 15.0 | 15.0 | 15.0 | 15.0 | 15.0 | 15.0 | 15.0 | 15.0 |
| | water | | | | | | | | | | | | | |
| | (volatile) | | | | | | | | | | | | | |
| Solvent | Ethanol | 200.0 | 200.0 | 200.0 | 200.0 | 200.0 | 200.0 | 200.0 | 200.0 | 200.0 | 200.0 | 200.0 | 200.0 | 200.0 |
| | (volatile) | | | | | | | | | | | | | |
| Solvent | Methylene | 200.0 | 200.0 | 200.0 | 200.0 | 200.0 | 200.0 | 200.0 | 200.0 | 200.0 | 200.0 | 200.0 | 200.0 | 200.0 |
| | chloride | | | | | | | | | | | | | |
| | (volatile) | | | | | | | | | | | | | |
| Total | | 165.0 | 165.0 | 165.0 | 164.0 | 120.0 | 100.0 | 110.0 | 120.0 | 120.0 | 120.0 | 120.0 | 123.0 | 117.0 |
| Film | Hydroxy- | 9.0 | 9.0 | 9.0 | 9.0 | 9.0 | 9.0 | 9.0 | 9.0 | 9.0 | 9.0 | 9.0 | 9.0 | 9.0 |
| coating | propyl- | | | | | | | | | | | | | |
| agent | methylcellulose | | | | | | | | | | | | | |
| | 2910 | | | | | | | | | | | | | |
| Plasticizer | Polyethylene- | 0.7 | 0.7 | 0.7 | 0.7 | 0.7 | 0.7 | 0.7 | 0.7 | 0.7 | 0.7 | 0.7 | 0.7 | 0.7 |
| | glycol | | | | | | | | | | | | | |
| | 6000 | | | | | | | | | | | | | |
| Coloring | Titanium | 0.3 | 0.3 | 0.3 | 0.3 | 0.3 | 0.3 | 0.3 | 0.3 | 0.3 | 0.3 | 0.3 | 0.3 | 0.3 |
| agent | oxide | | | | | | | | | | | | | |
| Film | Ethanol | 100.0 | 100.0 | 100.0 | 100.0 | 100.0 | 100.0 | 100.0 | 100.0 | 100.0 | 100.0 | 100.0 | 100.0 | 100.0 |
| coating | (volatile) | | | | | | | | | | | | | |
| solvent | | | | | | | | | | | | | | |
| Film | Methylene | 100.0 | 100.0 | 100.0 | 100.0 | 100.0 | 100.0 | 100.0 | 100.0 | 100.0 | 100.0 | 100.0 | 100.0 | 100.0 |
| coating | chloride | | | | | | | | | | | | | |
| solvent | (volatile) | | | | | | | | | | | | | |

| | | | | | | | | | | | | | |
|---|---|---|---|---|---|---|---|---|---|---|---|---|---|
| Total | 435.0 | 305.0 | 335.0 | 334.0 | 525.0 | 505.0 | 545.0 | 515.0 | 491.0 | 625.0 | 495.0 | 528.0 | 522.0 |
| Formulation for oral | multi- | multi- | multi- | multi- | inner | inner | inner | inner | inner | inner | inner | inner | inner |
| administration | layered | layered | layered | layered | core | core | core | core | core | core | core | core | core |
| | tablet | tablet | tablet | tablet | tablet | tablet | tablet | tablet | tablet | tablet | tablet | tablet | tablet |

### Comparative Example 1

### ARB Single Pill

Commercially available ARB drugs such as Cozaar® 50 mg tablet (MSD Korea, losartan single pill), Diovan® 80 mg tablet (Korea Novartis, valsartan single pill) and Pritor®40 mg tablet (GSK, telmisartan single pill) were used for the comparative study of the preparations according to the present invention.

### Comparative Example 2

### Amlodipine Single Pill

Commercially available dihydropyridine-based calcium channel blockers such as Anydipine® 5 mg tablet (CKD, amlodipine maleate single pill), Norvasc® 5 mg tablet (Pfizer, amlodipine besylate single pill), Zanidip® tablet (LG Life Science, lercanidipine hydrochloride) and Vaxar® 4 mg tablet (GSK, lacidipine single pill) were used for the comparative study of the preparations according to the present invention.

### Experimental Example 1

### Dissolution Test

Comparative dissolution tests were conducted based on the general dissolution test method described in Korea Pharmacopoeia (8th revision).

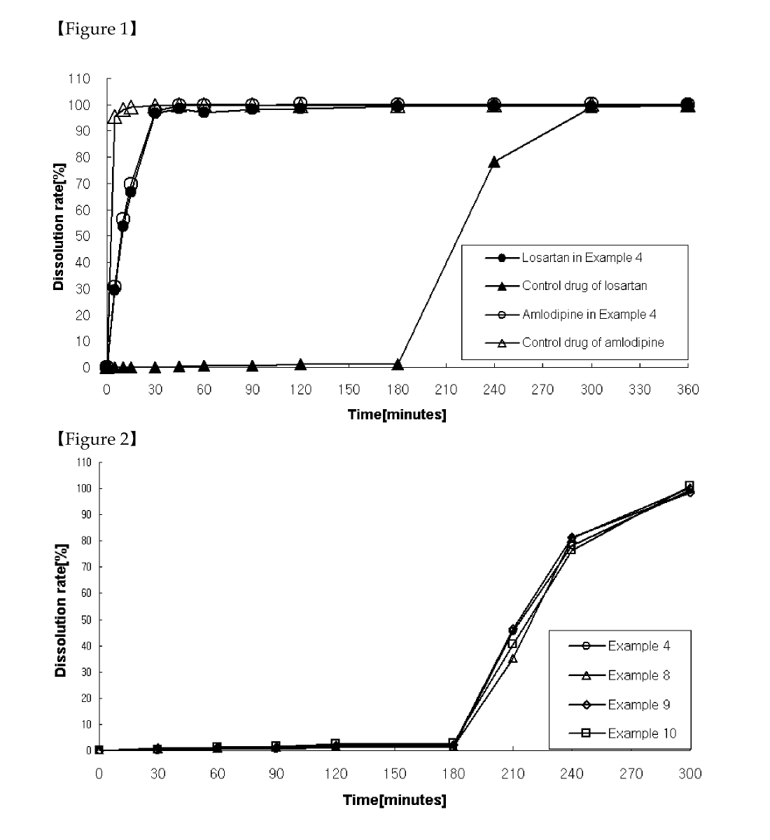

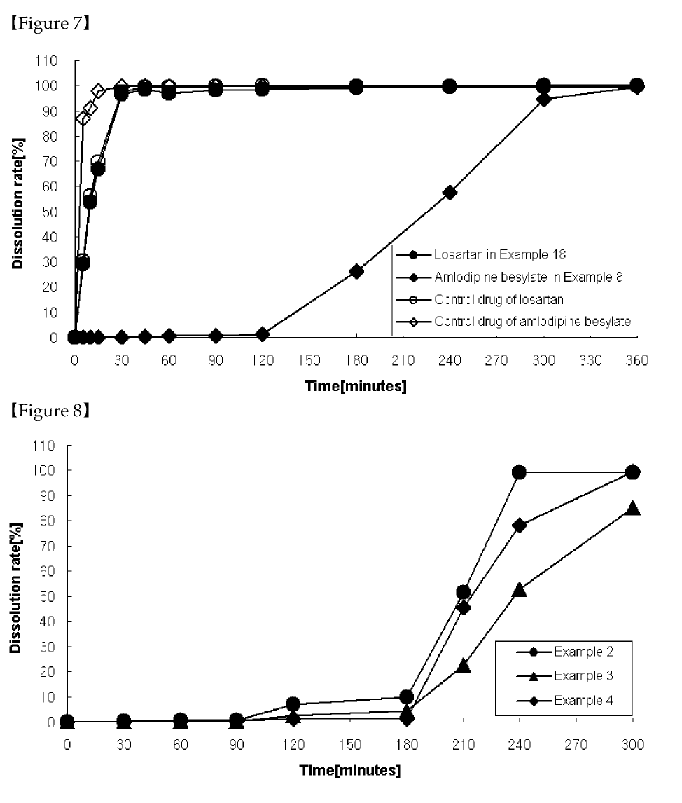

Dissolution test was performed with intestinal fluid without enzyme heated to 37±0.5° C.(Korea Pharmacopoeia 8^{th} revision; the second fluid in the disintegration test method) and in use of paddle method with the revolution of 50 rpm. The test for lercanidipine hydrochloride preparation was conducted by adding 1% of polysorbate 80 as a surfactant for dissolution test. Constant amount of dissolution liquid was separated at a predetermined time interval after the release, and the dissolution rates were analyzed, thus obtaining the results shown in FIGS. 1-8 (the number of each test agent is 12).

As shown in FIG. 1, losartan in a functional combination formulation of amlodipine-losartan was disintegrated from the beginning of the test as like a commercially available drug, and a dissolution rate increased to higher than 85% within 30 minutes in a solution of pH 6.8.

Unlike the commercially available drug, a delayed-immediate-release preparation of amlodipine started to be released 3-5 hours later after the beginning of the test as expected by the present inventors, and the dissolution rate increased to higher than 85% within an hour after amlodipine began to be released.

As shown in FIG. 2, amlodipine shows small variance in the dissolution rates depending on the formulations of administration. Therefore, it was ascertained that all the formulations for administration described in Examples may achieve the object of the present invention—losartan is first released and controls blood pressure during nighttime, while amlodipine is released a few hours later and controls blood pressure during daytime.

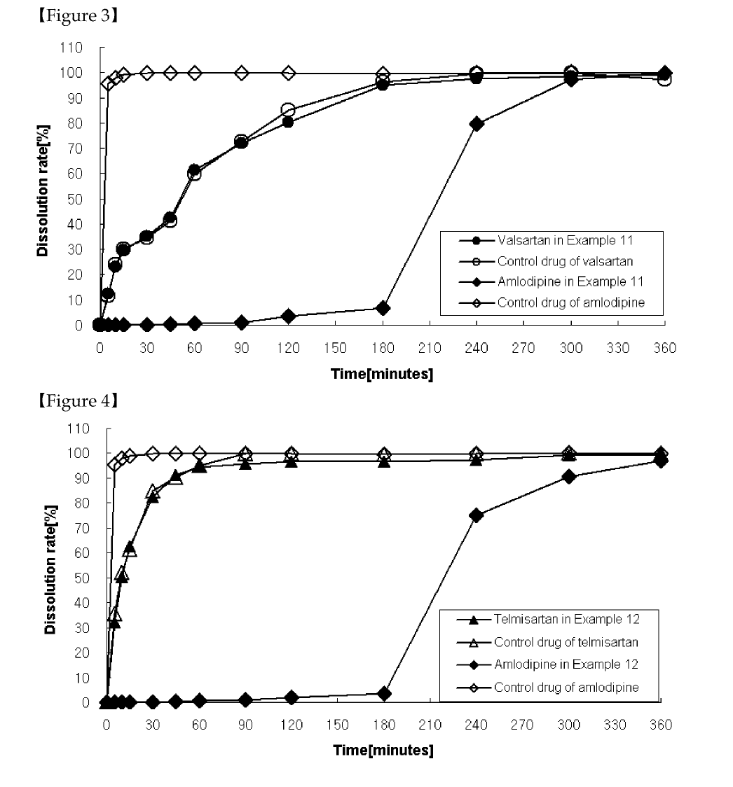

As shown in FIGS. 3 and 4, it was ascertained that other ARB based drugs (valsartan and telmisartan) may also be formulated according to the present invention, thereby achieving the object of the present invention although there is a little difference depending on solubility.

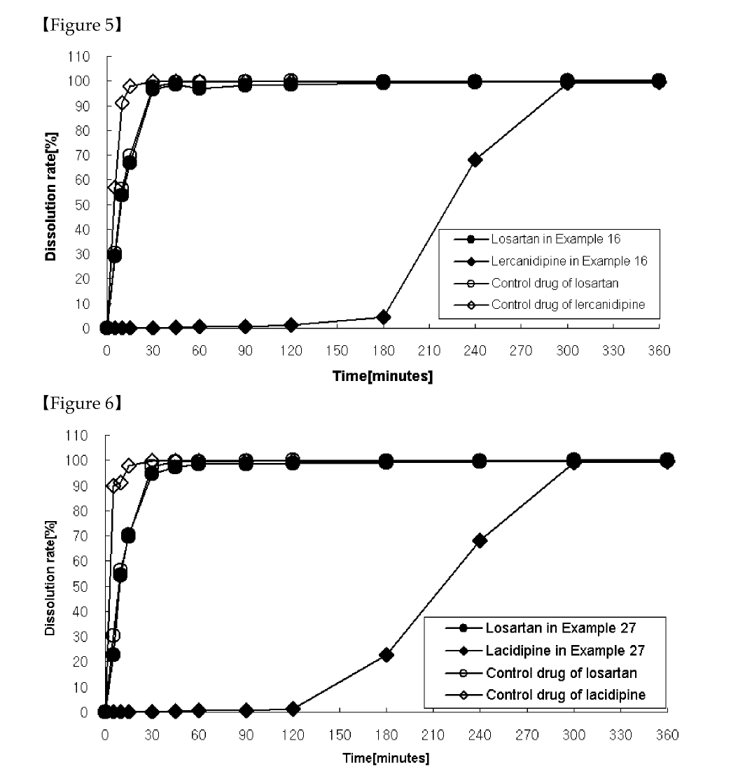

As shown in FIGS. 5-7, it was ascertained that other calcium channel blocker based drugs (lercanidipine hydrochloride and lacidipine) and other salts (amlodipine besylate) may be formulated along with losartan according to the present invention, thereby achieving the object of the present invention.

As shown in FIG. 8, it was ascertained that a delayed-immediate-release granule of amlodipine according to the present invention may also be prepared even by using one polymer of cellulose acetate.

### Experimental Example 2

### Efficacy Test (Animal Test)

Animal test was performed as described in Table 6 to compare the efficacy between an evening administration of a calcium channel blocker such as amlodipine and an ARB drug such as losartan and a chronotherapeutic administration with time interval in a tablet.

TABLE 6

| Title | Animal test for comparing the anti-hypertensive efficacy | | |
|---|---|---|---|
| | between a co-administration therapy and a chronotherapeutic | | |
| | therapy | | |
| Object | For obtaining optimum time and method of administration in a | | |
| | combination therapy of amlodipine and losartan, which are | | |
| | metabolized by the same enzyme | | |
| Test | Spontaneous Hypertensive Rat (SHR Rat) | | |
| animal | Wistar-Kyoto Rat (WKY Rat) | | |
| Test | Test group | Number | Administration route |
| method | Normal (WKY Rat, Saline) | 4 | p.o. |
| | Vehicle (Saline) | 6 | |
| | Amlodipine & Losartan | 5 | |
| | (Simultaneous, light) | | |
| | Amlodipine & Losartan | | |
| | (Simultaneous, dark) | | |
| | Amlodipine & Losartan | | |
| | (chronotherapeutic, light) | | |
| | Amlodipine & Losartan | | |
| | (chronotherapeutic, dark) | | |

| | 1. | Route and method of administration: Orally administered into | |
|---|---|---|---|
| | | the stomach by using a sonde and a syringe for oral | |
| | | administration. | |
| | 2. | Oral route was selected because the clinically administration | |
| | | route of the test material was expected as oral route. | |
| | 3. | Time of administration: | |
| | | 3.1 | simultaneous co-adminitration(co-administration) group: |
| | | | Both amlodipine and losartan were administered at 2 p.m. |
| | | 3.2 | chronotherapeutic administration group: Losartan and |
| | | | amlodipine were administered at 2 p.m. and 6 pm., respectively. |
| | 4. | Dosage(Administration dosage) | |
| | | 4.2 | Losartan: 50 mg/kg |
| | 5. | Volumetric amount of dosage: Dosage amount of 5 mL/kg | |
| | | was calculated based on the rat weight in the day of | |
| | | administration. | |
| | 6. | Frequency and period of administration: Once-daily | |
| | | administration for 5 consecutive days | |
| Eval- | 1. | Measurement of blood pressure | |
| uation | | 1.1 | Each rat was placed in a rat holder of blood pressure |
| method | | | monitor. |
| | | 1.2. | Blood pressure was measured by using a cuff-pulse sensor |
| | | | attached on a tail. |
| | | 1.3. | Blood pressure was measured 1-3 times, and an averaged |
| | | | value was recorded. |

| | |
|---|---|
| 2. | Item of measurement |
| | Heart Rate (HR), SBP (Systolic BP), MBP (Mean BP), DBP |
| | (Diastolic BP) |
| 3. | Time of measurement |

| | |
|---|---|
| 3.1. | Blood pressure was measured before administration. |
| 3.2. | Blood pressure was measured 20 hours later after |
| | administration. |
| 3.3. | Blood pressure was measured 1, 2 and 5 days later after |
| | administration. |

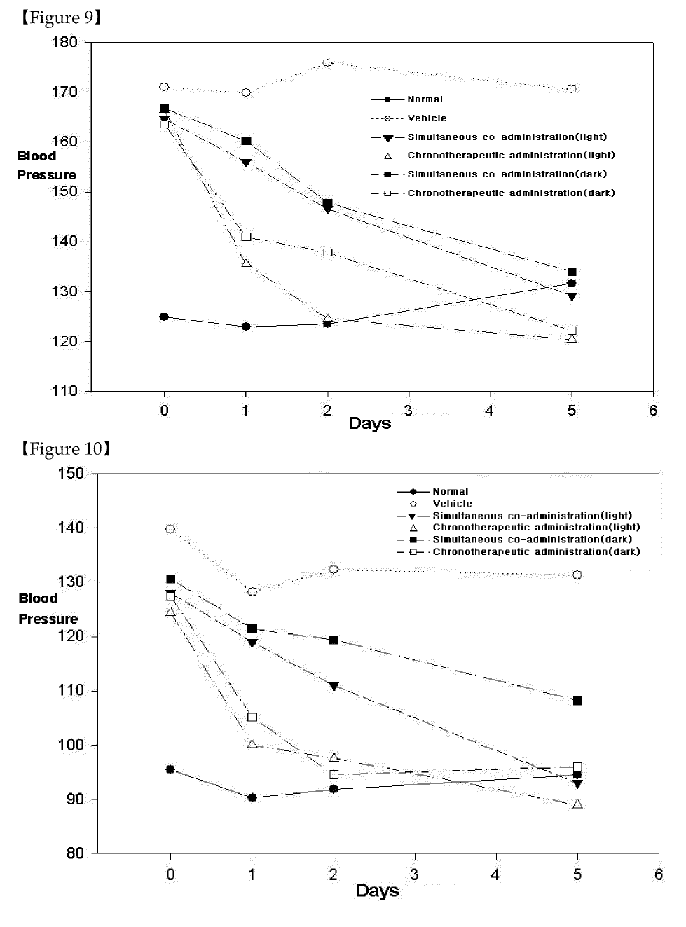

Referring to Table 7 and FIGS. 9-11, detailed description on the results of animal test are provided hereunder.

1. Blood pressure was significantly lowered on the day of administration only in a chronotherapeutic administration group (Table 7 and FIG. 9).

2. Administration under the light condition (same as administration in the evening for humans). showed lower blood pressure by more than about 15% than administration under the dark condition (same as administration in the morning for humans).

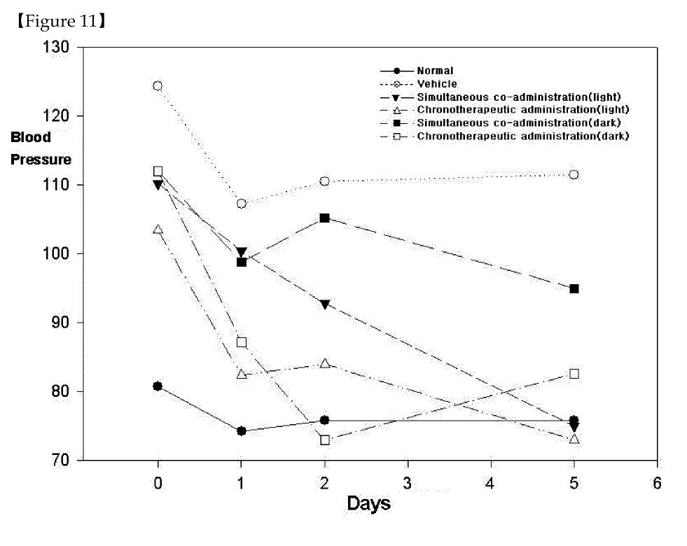

3. All the test groups showed a significantly lowered blood pressure on the 2^{nd} and 5^{th} day of administration (Table 7 and FIGS. 10 and 11).

4. A chronotherapeutic administration group (administration in the evening) according to the present invention is the highest in the effect of lowering blood pressure, followed by a chronotherapeutic administration group (administration in the morning), a co-administration group(administration in the evening) and a co-administration group(administration in the morning).

As described above, a chronotherapeutic administration group is superior to a co-administration group in inhibiting the increase of blood pressure, which may be explained by xenobiotics and chronotherapy as disclosed in the present invention. That is, amlodipine inhibits the activity of hepatic metabolizing enzyme (cytochrome P450 3A4) in a co-administration group, thereby antagonizing the conversion of losartan into an activated form. However, in a chronotherapeutic administration group, amlodipine is released or absorbed after losartan is converted into an activated form, thereby showing relatively higher activity of inhibiting the increase of blood pressure.

In a combination therapy of amlodipine and losartan, it was ascertained that the administration in the evening is higher than the administration in the morning in the therapeutic activity. This is because losartan is preferred to be administered in the evening as the production of renin, a hypertension inducer, is increased in one's sleep. Further, a chronotherapeutic administration group was ascertained as superior activity of controlling blood pressure at the beginning of administration. Therefore, when a combination preparation of amlodipine and losartan is used for the treatment of hypertension, a chronotherapeutic administration in the evening (amlodipine administration after losartan administration) was ascertained as optimum therapy for lowering blood pressure.

TABLE 7
Blood pressure at 20 hours later after the administration of drug

| | | | | |
|---|---|---|---|---|
| Group | HR | SBP | MBP | DBP |

0 Day

| | | | | |
|---|---|---|---|---|
| Normal | 471.8 ± 45.5 | 125.0 ± 7.5 | 95.5 ± 9.7 | 80.8 ± 12.6 |
| Vehicle | 477.7 ± 45.3 | 171.0 ± 8.7 | 139.0 ± 12.4 | 124.3 ± 15.5 |
| Amlodipine & Losartan | 499.2 ± 49.6 | 164.6 ± 15.3 | 128.0 ± 11.7 | 110.2 ± 11.8 |
| (Simultaneous, light) | | | | |
| Amlodipine & Losartan | 424.4 ± 58.3 | 166.6 ± 14.0 | 130.6 ± 12.6 | 112.0 ± 12.4 |
| (Simultaneous, dark) | | | | |
| Amlodipine & Losartan | 480.6 ± 72.7 | 166.4 ± 7.1 | 124.4 ± 7.2 | 103.4 ± 8.4 |
| (Chronotherapeutic, light) | | | | |
| Amlodipine & Losartan | 418.2 ± 38.9 | 163.6 ± 8.7 | 127.4 ± 12.9 | 109.2 ± 19.7 |
| (Chronotherapeutic, dark) | | | | |

1 Day

| | | | | |
|---|---|---|---|---|
| Normal | 487.8 ± 25.8 | 123.0 ± 6.2 | 90.3 ± 14.5 | 74.3 ± 19.9 |
| Vehicle | 473.3 ± 46.1 | 169.8 ± 15.3 | 128.2 ± 11.1 | 107.2 ± 13.0 |
| Amlodipine & Losartan | 466.2 ± 39.0 | 156.0 ± 17.7 | 119.0 ± 13.8 | 100.4 ± 16.4 |
| (Simultaneous, light) | | | | |
| Amlodipine & Losartan | 456.8 ± 18.1 | 160.2 ± 5.7 | 121.4 ± 14.2 | 98.8 ± 16.8 |
| (Simultaneous, dark) | | | | |
| Amlodipine & Losartan | 442.0 ± 74.1 | 135.6 ± 19.7** | 100.0 ± 13.2** | 82.4 ± 13.7* |
| (Chronotherapeutic, light) | | | | |
| Amlodipine & Losartan | 486.4 ± 48.2 | 141.0 ± 11.2** | 105.2 ± 12.3** | 87.2 ± 15.4* |
| (Chronotherapeutic, dark) | | | | |

2 Day

| | | | | |
|---|---|---|---|---|
| Normal | 486.3 ± 35.6 | 123.5 ± 10.8 | 91.8 ± 7.4 | 75.8 ± 10.6 |
| Vehicle | 477.2 ± 27.5 | 175.6 ± 8.3 | 132.3 ± 10.9 | 110.5 ± 13.0 |
| Amlodipine & Losartan | 444.2 ± 83.4 | 146.6 ± 3.0** | 111.0 ± 4.9** | 92.8 ± 7.5* |
| (Simultaneous, light) | | | | |
| Amlodipine & Losartan | 432.2 ± 20.4 | 147.8 ± 17.8** | 119.4 ± 13.3 | 105.2 ± 12.4 |
| (Simultaneous, dark) | | | | |
| Amlodipine & Losartan | 428.8 ± 111.4 | 124.6 ± 6.4** | 97.6 ± 16.1** | 84.0 ± 23.1* |
| (Chronotherapeutic, light) | | | | |
| Amlodipine & Losartan | 468.2 ± 23.1 | 137.8 ± 9.4** | 94.6 ± 10.6** | 73.0 ± 13.6** |
| (Chronotherapeutic, dark) | | | | |

5 Day

| | | | | |
|---|---|---|---|---|
| Normal | 460.0 ± 37.1 | 131.8 ± 3.8 | 94.5 ± 6.6 | 75.8 ± 8.5 |
| Vehicle | 453.8 ± 77.2 | 170.5 ± 9.2 | 131.3 ± 12.6 | 111.5 ± 19.2 |
| Amlodipine & Losartan | 426.6 ± 42.0 | 129.2 ± 4.3** | 93.0 ± 2.0** | 75.0 ± 3.5* |
| (Simultaneous, light) | | | | |
| Amlodipine & Losartan | 440.4 ± 18.3 | 134.0 ± 15.3** | 108.2 ± 13.4* | 95.0 ± 14.5 |
| (Simultaneous, dark) | | | | |
| Amlodipine & Losartan | 510.2 ± 20.1 | 120.4 ± 3.9** | 89.0 ± 6.6** | 73.0 ± 10.3* |
| (Chronotherapeutic, light) | | | | |
| Amlodipine & Losartan | 427.0 ± 74.2 | 122.2 ± 13.8** | 96.0 ± 14.6* | 82.6 ± 15.5 |
| (Chronotherapeutic, dark) | | | | |

### INDUSTRIAL APPLICABILITY

As described above, a functional combination preparation herein fully achieves the pharmaceutical and clinical efficacy that may not be accomplished by a co-administration of an amlodipine single pill and a losartan single pill by applying xenobiotics and chronotherapy theory to drug formulation design. Further, a functional combination preparation herein may show constant activities of inhibiting the increase in blood pressure and preventing complications because it may be administered in the evening. A simple medication instruction may increase the compliance especially for aged peoples.

Further, it is expected that a functional combination preparation will increase the preventive or therapeutic activity for mild hypertension up to about 80% from about 50% in single pills. It contributes to the longevity of the hypertensive patients that such a functional combinations preparation shows remarkable efficacy for three major complications of heart disease, kidney disease and stroke.

In particular, a functional combination preparation will be the best prescription or therapy for a hypertensive patient suffering from diabetes complication.

Further, the two drugs in a functional combination preparation herein have different activities and reduce the side effects of each drug, and also lower the risk of circulatory complications. The present invention is also efficient in economical respect in that a combination prescription will curtail the long-term expenses to be incurred for the prevention of diseases, the package cost for each single pill and the prescription time.

Therefore, the present invention will open the new aera of a functional combination preparation by applying xenobiotics and chronotherapy theory to the drug formulation technique.

1. A functional combination preparation comprising a dihydropyridine-based calcium channel blocker and an angiotensin-2 receptor blocker (ARB) as active ingredients, wherein the angiotensin-2 receptor blocker (ARB) is rapidly released while the dihydropyridine-based calcium channel blocker is released after some lag time.

2. The functional combination preparation of claim 1, wherein the release of the dihydropyridine-based calcium channel blocker is delayed for 1-6 hours so that the dihydropyridine-based calcium channel blocker may be absorbed after metabolism of the angiotensin-2 receptor blocker (ARB).

3. The functional combination preparation of claim 1, which comprises:
an immediate-release part comprising the angiotensin-2 receptor blocker (ARB) as an active ingredient; and
a delayed-immediate-release part comprising the dihydropyridine-based calcium channel blocker as an active ingredient and a release-controlling material selected from the group consisting of a water-soluble polymer, a water-insoluble polymer, an enteric polymer and a mixture thereof.

4. The functional combination preparation of claim 1, wherein the dihydropyridine-based calcium channel blocker is selected from the group consisting of amlodipine, lercanidipine, felodipine, nifedipine, nicardipine, isradipine, nisoldipine or a pharmaceutically acceptable salts thereof.

5. The functional combination preparation of claim 1, wherein the angiotensin-2 receptor blocker (ARB) is selected from the group consisting of losartan, valsartan, telmisartan, irbesartan, candesartan, olmesartan or pharmaceutically acceptable salts.

6. A functional combination preparation for the treatment of cardiovascular disease comprising:
1) an immediate-release granule comprising an angiotensin-2 receptor blocker (ARB) as an active ingredient; and
2) a delayed-immediate-release granule or coated tablet comprising a dihydropyridine-based calcium channel blocker as active ingredients and a release-controlling material selected from the group consisting of a water-soluble polymer, a water-insoluble polymer, an enteric polymer and a mixture thereof.

7. The functional combination preparation of claim 6, wherein the release of the dihydropyridine-based calcium channel blocker is delayed for 1-6 hours so that the dihydropyridine-based calcium channel blocker may be absorbed after metabolism of the angiotensin-2 receptor blocker (ARB).

8. The functional combination preparation of claim 6, wherein the dihydropyridine-based calcium channel blocker is selected from the group consisting of amlodipine, lercanidipine, felodipine, nifedipine, nicardipine, isradipine, nisoldipine or pharmaceutically acceptable salts thereof.

9. The functional combination preparation of claim 6, wherein the dihydropyridine-based calcium channel blocker is amlodipine or a pharmaceutically acceptable salts thereof.

10. The functional combination preparation of claim 6, wherein the angiotensin-2 receptor blocker (ARB) is selected from the group consisting of losartan, valsartan, telmisartan, irbesartan, candesartan, olmesartan or pharmaceutically acceptable salts thereof.

11. The functional combination preparation of claim 6, wherein the angiotensin-2 receptor blocker (ARB) is losartan or pharmaceutically acceptable salts thereof.

12. The functional combination preparation of claim 6, wherein the angiotensin-2 receptor blocker (ARB) is contained in the amount of 0.2-20 weight parts relative to one weight part of the dihydropyridine-based calcium channel blocker.

13. The functional combination preparation of claim 6, wherein the release-controlling material is contained in the amount of 0.5-100 weight parts relative to one weight part of the dihydropyridine-based calcium channel blocker.

14. The functional combination preparation of claim 6, wherein the water-soluble polymer is selected from the group consisting of a water-soluble cellulose ether selected from the group consisting of methylcellulose, hydroxypropylcellulose and hydroxypropylmethylcellulose; a water-soluble polyvinyl derivative selected from the group consisting of polyvinylpyrrolidone and polyvinylalcohol; an alkylene oxide polymer selected from the group consisting of polyethylene glycol and polypropylene glycol; and a mixture thereof.

15. The functional combination preparation of claim 6, wherein the water-insoluble polymer is a water-insoluble cellulose ether selected from the group consisting of ethylcellulose and cellulose acetate; a water-insoluble acrylic acid based copolymer acrylic acid ethyl.methacrylic acid methyl.methacrylic acid chlorotrimethylammonium ethyl copolymer and methacrylic acid methyl.acrylic acid ethyl copolymer chlorotrimethylammonium ethyl copolymer; and a mixture thereof.

16. The functional combination preparation of claim 6, wherein the enteric polymer is selected from the group consisting of an enteric cellulose derivative selected from the group consisting of hydroxypropylmethylcellulose acetate succinate, hydroxypropylmethylcellulose phthalate, hydroxymethylethylcellulose phthalate, cellulose acetate phthalate, cellulose acetate succinate, cellulose acetate maleate, cellulose benzoate phthalate, cellulose propionate phthalate, methylcellulose phthalate, carboxymethylethylcellulose and ethylhydroxyethylcellulose phthalate; an enteric acrylic acid based copolymer selected from the group consisting of styrene.acrylic acid copolymer, acrylic acid methyl.acrylic acid copolymer, acrylic acid methylmethacrylic acid copolymer, acrylic acid butyl.styrene.acrylic acid copolymer, methacrylic acid.methacrylic acid ethyl copolymer, methacrylic acid.acrylic acid ethylcopolymer and acrylic acid methyl.methacrylic acid.acrylic acid octylcopolymer; an enteric maleic acid based copolymer selected from the group consisting of acetic acid vinyl.maleic acid anhydride copolymer, styrene.maleic acid anhydride copolymer, styrene.maleic acid monoester copolymer, vinylmethylether.maleic acid anhydride copolymer, ethylene.maleic acid anhydride copolymer, vinylbutylether.maleic acid anhydride copolymer, acrylonitrile.acrylic acid methyl.maleic acid anhydride copolymer and acrylic acid butyl.styrene.maleic acid anhydride copolymer; an enteric polyvinyl derivative selected from the group consisting of polyvinylalcohol phthalate, polyvinylacetal phthalate, polyvinylbutyrate phthalate and polyvinylacetoacetal phthalate; and a mixture thereof.

17. The functional combination preparation of claim 6, wherein the functional combination preparation is formulated into a form selected from the group consisting of an uncoated tablet, a coated tablet having a film coating layer, a multi-layered tablet, an inner core tablet, powders, granules and a capsule.

18. The functional combination preparation of claim 17, wherein the multi-layered tablet comprises a dihydropyridine-based calcium channel blocker layer that is immediately released after some lag time; and an angiotensin-2 receptor blocker (ARB) layer that is immediately released.

19. The functional combination preparation of claim 18, wherein the release of the dihydropyridine-based calcium channel blocker is delayed for 1-6 hours so that the dihydropyridine-based calcium channel blocker may be absorbed after metabolism of the angiotensin-2 receptor blocker (ARB).

20. The functional combination preparation of claim 17, wherein the inner core tablet comprises a core tablet of dihydropyridine-based calcium channel blocker that is immediately released after some lag time; and an outer layer of angiotensin-2 receptor blocker (ARB) that is immediately released.

21. The functional combination preparation of claim 17, wherein the capsule comprises a granule of dihydropyridine-based calcium channel blocker that is immediately released after some lag time; and a granule of angiotensin-2 receptor blocker (ARB) that is immediately released.

22. The functional combination preparation of claim 17, wherein the coating layer comprises a film former, a film-forming adjuvant or a mixture thereof.

23. The functional combination preparation of claim 17, wherein the dihydropyridine-based calcium channel blocker and the ARB (angiotensin-2 receptor blocker) in a preparation in the amount of 2.5-30 mg and 12.5-300 mg, respectively.
